(19) United States
(12) Patent Application Publication
(10) Pub. No.: US 2026/0102566 A1
(43) Pub. Date: Apr. 16, 2026

(54) LASER FREE TATTOO REMOVAL SYSTEM

(71) Applicant: LuX Tattoo Removal, LLC, Wooster, OH (US)

(72) Inventors: Jillian Marty, Wooster, OH (US); Chad Novak, Uniontown, OH (US)

(73) Assignee: LuX Tattoo Removal, LLC, Wooster, OH (US)

(21) Appl. No.: 19/355,484

(22) Filed: Oct. 10, 2025

Related U.S. Application Data
(60) Provisional application No. 63/706,937, filed on Oct. 14, 2024.

(51) Int. Cl. A61M 5/19; A61M 5/145; A61M 5/31; A61M 5/315; A61M 5/32; A61M 5/42

(52) U.S. Cl. CPC A61M 5/19; A61M 5/1452; A61M 5/3129; A61M 5/31541; A61M 5/3298; A61M 5/422; A61M 2205/18; A61M 2205/3576; A61M 2205/505; A61M 2210/04

(57) ABSTRACT

A laser-free method of tattoo and/or permanent makeup removal, including a laser-free tattoo removal pen, software of the tattoo removal pen, a needle cartridge, at least one needle bundle, a numbing solution, a tattoo removal solution, a syringe containing the numbing solution, a syringe containing the tattoo removal solution, a dual syringe infusion pump, and medical-grade aftercare products with an individualized aftercare plan. The dual syringe pump is operable to simultaneously administer the numbing solution and the tattoo removal solution to the treatment area of skin of a human patient through a Y-splitter structure and out through the needle bundle of the laser-free tattoo removal pen.

### CROSS REFERENCES TO RELATED APPLICATIONS

This application claims priority to and the benefit of U.S. Application No. 63/706,937 filed on Oct. 14, 2024, which is incorporated herein by reference in its entirety.

### BACKGROUND OF THE INVENTION

### 1. Field of the Invention

The present invention relates to tattoo and permanent makeup removal, and more specifically to non-laser methods of tattoo and permanent makeup removal including providing simultaneous numbing and removal of the tattoo or permanent makeup via a laser-free tattoo removal pen.

### 2. Description of the Prior Art

It is generally known in the prior art to provide methods for tattoo removal including laser removal, surgical excision, dermabrasion, chemical peels, saline removal, and/or removal creams.

Prior art patent documents include the following:

U.S. Pat. No. 10,500,013 for Method for removing a tattoo through patterned trans-epidermal pigment release by inventor Turner, et al., filed Sep. 9, 2016, and issued Dec. 10, 2019, is directed to embodiments of a method for removing a tattoo through patterned trans-epidermal pigment release including determining first treatment area of skin of a patient through a primary template including primary apertures, marking the first treatment area of skin of the patient along borders of the primary apertures to outline a grid of primary tegulae, and delivering a tattoo removal fluid to the marked first exposed skin in a first treatment session. The method further includes determining a secondary treatment area through a secondary template including secondary apertures, marking the secondary treatment area along borders of the secondary apertures to outline a grid of secondary tegulae, and delivering a tattoo removal fluid to the secondary tegulae in a second treatment session.

U.S. Pat. No. 11,771,877 for Method for removing a tattoo through patterned trans-epidermal pigment release by inventor Turner, et al., filed Dec. 9, 2019, and issued Oct. 3, 2023, is directed to embodiments of a method for removing a tattoo through patterned trans-epidermal pigment release including determining first treatment area of skin of a patient through a primary template including primary apertures, marking the first treatment area of skin of the patient along borders of the primary apertures to outline a grid of primary tegulae, and delivering a tattoo removal fluid to the marked first exposed skin. A template, which may be adhered to the skin, is used during a microneedling process to create a structured, patterned procedure to remove skin irregularities. The template has a plurality of needle apertures, an adhesive layer, and a release liner. The release liner may be removed, exposing the adhesive layer, so that the template, with the plurality of needle apertures, may be positioned over the skin irregularity.

US Patent Publication No. 2021/0113296 for Template and method for removing a tattoo through patterned trans-epidermal pigment release by inventors Savage, et al., filed Dec. 8, 2020, and published Apr. 22, 2021, is directed to a method for removing a tattoo through patterned trans-epidermal pigment release including determining first treatment area of skin of a patient through a primary template including primary apertures, marking the first treatment area of skin of the patient along borders of the primary apertures to outline a grid of primary tegulae, and delivering a tattoo removal fluid to the marked first exposed skin. A template, which may be adhered to the skin, is used during a disruption process to create a structured, patterned procedure to remove skin irregularities. The template has a plurality of needle apertures, an adhesive layer, and a release liner. The release liner may be removed, exposing the adhesive layer, so that the template, with the plurality of needle apertures, may be positioned over the skin irregularity.

US Patent Publication No. 2021/0106398 for Template and method for removing a tattoo through patterned trans-epidermal pigment release by inventors Turner, et al., filed Dec. 8, 2020, and published Apr. 15, 2021, is directed to a method for removing a tattoo through patterned trans-epidermal pigment release including determining first treatment area of skin of a patient through a primary template including primary apertures, marking the first treatment area of skin of the patient along borders of the primary apertures to outline a grid of primary tegulae, and delivering a tattoo removal fluid to the marked first exposed skin. A template, which may be adhered to the skin, is used during a disruption process to create a structured, patterned procedure to remove skin irregularities. The template has a plurality of needle apertures, an adhesive layer, and a release liner. The release liner may be removed, exposing the adhesive layer, so that the template, with the plurality of needle apertures, may be positioned over the skin irregularity.

European Patent Publication No. 4,201,345 for Method for removing a tattoo through patterned trans-epidermal pigment release by inventors Turner, et al., filed Dec. 8, 2020, and published Jun. 28, 2023, is directed to a method for removing a tattoo through patterned trans-epidermal pigment release including determining first treatment area of skin of a patient through securing a primary template including primary apertures. The first treatment area is then marked along borders of the primary apertures to outline a grid of primary tegulae, disrupting the tissue in the primary tegulae to form an eschar in a first treatment session. A second treatment area is determined by marking secondary tegulae interposed between primary tegulae and disrupting same.

U.S. Pat. No. 11,020,203 for System for puncturing the epidermis with controlled fluid delivery by inventors Niven, et al., filed Sep. 9, 2016, and issued Jun. 1, 2021, is directed to an actuating device for actuating a needle cartridge and puncturing an epidermis with controlled depth. The actuating device includes a motor housed in a motor housing, an actuating rod driven by the motor and configured to actuate in a reciprocating motion, and an adjustment mechanism. The actuating rod is housed in a rod housing, the rod housing forming a device aperture configured to receive a needle cartridge and to attach to the needle cartridge. The adjustment mechanism interfaces with the rod housing and is configured to adjust a position of the rod housing relative to the motor housing while not rotating the rod housing relative to the motor housing.

U.S. Pat. No. 11,986,615 for System for puncturing the epidermis with controlled fluid delivery by inventors Niven, et al., filed Apr. 30, 2021, and issued May 21, 2024, is directed to an actuating device for actuating a needle cartridge and puncturing an epidermis with controlled depth. The actuating device includes a motor housed in a motor housing, an actuating rod driven by the motor and configured to actuate in a reciprocating motion, and an adjustment mechanism. The actuating rod is housed in a rod housing, the rod housing forming a device aperture configured to receive a needle cartridge and to attach to the needle cartridge. The adjustment mechanism interfaces with the rod housing and is configured to adjust a position of the rod housing relative to the motor housing while not rotating the rod housing relative to the motor housing.

U.S. Pat. No. 10,610,327 for Apparatus and method for puncturing the epidermis with controlled fluid delivery by inventors Niven, et al., filed Sep. 9, 2016, and issued Apr. 7, 2020, is directed to an apparatus including a needle cartridge configured to attach to and interface with an actuating device. The needle cartridge includes a housing, a needle group assembly, and a fluid port. The housing forms a cavity and includes a first aperture and a second aperture opposite the first aperture. The needle group assembly includes a needle and a needle holder. The needle group assembly is configured to move in a reciprocating motion relative to the housing along an axis from the first aperture to the second aperture. During the reciprocating motion, the needle fully retracts into the cavity through the first aperture. The fluid port is integrated with the housing and includes a hollow projection forming a conduit and extending away from the housing. The fluid port includes a third aperture, wherein the fluid port is configured to interface with a fluid delivery system.

U.S. Pat. No. 11,529,505 for Apparatus and method for puncturing the epidermis with controlled fluid delivery by inventors Niven, et al., filed Mar. 27, 2020, and issued Dec. 20, 2022, is directed to an apparatus including a needle cartridge configured to attach to and interface with an actuating device. The needle cartridge includes a housing, a needle group assembly, and a fluid port. The housing forms a cavity and includes a first aperture and a second aperture opposite the first aperture. The needle group assembly includes a needle and a needle holder. The needle group assembly is configured to move in a reciprocating motion relative to the housing along an axis from the first aperture to the second aperture. During the reciprocating motion, the needle fully retracts into the cavity through the first aperture. The fluid port is integrated with the housing and includes a hollow projection forming a conduit and extending away from the housing. The fluid port includes a third aperture, wherein the fluid port is configured to interface with a fluid delivery system.

US Patent Publication No. 2023/0211139 for System in use in the application and removal of tattoos and other skin treatments by inventors Niven, et al., filed Dec. 30, 2022, and published Jul. 6, 2023, is directed to an automated system featuring a system controller, an oscillation drive, a linear drive, a rotational drive, a flexible drive shaft, a handpiece, and a disposable cartridge used for the application or removal of tattoos and for use in skin treatment such as needling, skin tightening, aesthetic microneedling, and superficial dermabrasion procedures. A needle cartridge or plurality of blades may be couplable in modular configurations within the disposable cartridge. The automated system may also have a plurality of blades or needle bundles in various configurations couplable to the disposable cartridge selected in accordance to the skin treatment performed. The system may also include a physically integrated and functionally automated fluid pump with associated disposable fluid container and disposable feed tube. Alternately, the fluid pump may stand physically and functionally independent of the automated system.

US Patent Publication No. 2023/0211138 for System for use in the application and removal of tattoos and other skin treatments by inventors Niven, et al., filed Dec. 30, 2022, and published Jul. 6, 2023, is directed to an automated system featuring a system controller, an oscillation drive, a linear drive, a rotational drive, a flexible drive shaft, a handpiece, and a disposable cartridge used for the application or removal of tattoos and for use in skin treatment such as needling, skin tightening, aesthetic microneedling, and superficial dermabrasion procedures. A needle cartridge or plurality of blades may be couplable in modular configurations within the disposable cartridge. The automated system may also have a plurality of blades or needle bundles in various configurations couplable to the disposable cartridge selected in accordance to the skin treatment performed. The system may also include a physically integrated and functionally automated fluid pump with associated disposable fluid container and disposable feed tube. Alternately, the fluid pump may stand physically and functionally independent of the automated system.

U.S. Pat. No. 8,663,162 for Tattoo removal system by inventors Bunting, et al., filed Jul. 27, 2012, and issued Mar. 4, 2014, is directed to a system and associated materials and methods for delivering a fluid to a subject's skin, e.g., for tattoo removal. The fluid is provided in a continuous flow by associating the fluid with a pump. A disposable template is provided that allows for precise and ideal placement of eschar-inducing material fluid for, for example, tattoo removal.

US Patent Publication No. 2024/0180773 for Prediction, simulation, and analysis tool for mechanical disruption of soft tissues by inventors Turner, et al., filed Dec. 6, 2023, and published Jun. 6, 2024, is directed to mechanical disruption of soft tissues is a treatment modality for many dermatological, cosmetic, and medical procedures that has defied quantification. When driven through soft tissue, sturdy mechanical implements are not affected by the tissue, which permits a useful separation of specified kinematics from unknown dynamics. Tissue disruption (breaking structural fibers and other bonds) is proportional to the mechanical energy density delivered to the tissues, which in turn is related to a novel quantity: the kinematic displacement density (“KDD”). A kinematic displacement tool, including software with auxiliary hardware, may be used for, among other purposes, calculating, displaying, and using KDD in various application categories, including but not limited to: simulation and analysis of mechanical tissue disruption for medical purposes; training disruption technicians; optimizing disruption implements; programming semi-automatic disruption devices; and measuring the actual mechanical energy density delivered to tissues.

US Patent Publication No. 2021/0220632 for Non-laser tattoo removal method by inventors Piroli, et al., filed Jan. 21, 2021, and published Jul. 22, 2021, is directed to a non-laser tattoo removal method that includes the steps of cleaning and drying a target area on a user's skin, the target area including at least one tattoo disposed thereon; administering a deracination serum to the target area with a handheld automatic needle assembly having a plurality of needles after the cleaning and drying of the target area; wiping the target area with an absorbent material to remove an extracted pigmented material at least partially composing the tattoo; applying a malleable clay formulation material to cover the target area and leaving the malleable clay formulation material on the target area until forming a dried-on clay formulation; removing the dried-on clay formulation from the target area; and applying a liquid healing agent to the target area after removing the dried-on clay formulation from the target area and covering the liquid healing agent with at least one transdermal drug delivery sheet for selective removal by the user after a healing time period.

US Patent Publication No. 2010/0247687 for Formulation for tattoo removal and method of using the same by inventor Arnold-Ronish, filed Mar. 30, 2009, and published Sep. 30, 2010, is directed to a formulation for tattoo removal and method for using the same.

U.S. Pat. No. 11,197,813 for Tattoo removal by inventors Johnson, et al., filed May 14, 2018, and issued Dec. 14, 2021, is directed to tattoo removal. In an example, a method for removing tattoo ink from skin or resolving a medical condition related to tattoo ink, includes applying a primary solution of beta glucan to a treatment area having tattooed skin. The primary solution stimulates the skin in the treatment area to enhance removal of the tattoo ink from the skin. The example method also includes coating the treatment area with a secondary solution. The secondary solution may have an absorbent material to draw in loose ink. The example method also includes disrupting a skin barrier in the treatment area by a needle or ablative device to improve penetration of at least one of the primary solution and the secondary solution.

US Patent Publication No. 2017/0120071 for System, apparatus, and method of providing tattoo fading and removal by inventor Dabney, filed Oct. 28, 2016, and published May 4, 2017, is directed to a retainer, system and method of tattoo removal including treating a tattooed region of skin with an oxidizing or bleaching solution activated by certain wavelengths of light. The method of tattoo removal may include applying a layer of removal solution to a tattooed region of skin; regulating a temperature of the layer of removal solution with a temperature control element; and exposing the layer of removal solution to a light source of pre-determined wavelength. Light of certain wavelengths may improve or “supercharge” the effects of certain pharmaceuticals or target chemicals and agents, creating a synergistic effect that may break down tattoo ink molecules and eliminate the appearance of tattoos in the skin of humans or other animals.

US Patent Publication No. 2015/0375014 for Methods and systems for tattoo removal by inventors Slayton, et al., filed Jun. 26, 2015, and published Dec. 31, 2015, is directed to a system and method for tattoo removal utilizing controlled acoustic energy deposition. The system and method can generate an acousto-mechanical or acousto-elastic effect in tattoo pigment particles or agglomerates of pigment particles. The acousto-mechanical or acousto-elastic effect can induce a fragmentation of the pigment particles or agglomerates of pigment particles, creating sub-particles of a size which can be less visible from the surface of the skin or cleared from dermal tissue entirely.

U.S. Pat. No. 9,901,724 for Skin puncturing apparatus for use in a non-surgical method of eradication of tattoos by inventor Danenberg, filed Dec. 9, 2013, and issued Feb. 27, 2018, is directed to a skin puncturing apparatus for use in a non-surgical method for eradication of a tattoo from an area of skin. The apparatus comprises: (a) a handle section comprising a motor and gear assembly for causing a reciprocating motion to a shaft connected to the gear assembly; (b) a barrel section surrounding the shaft, the first end of the barrel section attached to the handle and the second end of the barrel section having a tip adapted to be placed in contact with the area of skin; (c) a bundle of needles attached by means of a needles holder to the distal end of the shaft; and (d) an inlet body comprising an inlet port through which cleaning solution is introduced into the tip located at the lower edge of the barrel section.

U.S. Pat. No. 9,364,650 for Method for removing pigments from a pigmented section of skin by inventors Hazut, et al., filed Dec. 8, 2005, and issued Jun. 14, 2016, is directed to a method for removing pigments from a pigmented section of a skin by puncturing the skin at the pigmented section, with a skin puncturing device which is provided with at least one needle, and then bandaging the punctured section with a suitable adsorbing pad. The pad contains one or more materials, such as saline, which will cause the pigments at the punctured section to migrate into the outer layer of the skin.

US Patent Pub. No. 2007/0166252 for Method of treating skin requiring tattoo removal by inventors Hattendorf, et al., filed Dec. 29, 2006, and published Jul. 19, 2007, is directed to a treatment regimen for treating skin subject to tattoo removal procedures involving the application of supplemental composition(s) such as preparatory composition(s), protective composition(s), and combinations thereof, and a corrective composition.

U.S. Pat. No. 6,773,698 for Tattoo removal by inventors Melinte, et al., filed Jun. 9, 2003, and issued Aug. 10, 2004, is directed to a three solution application method for tattoo removal.

U.S. Pat. No. 6,699,509 for Tattoo removal by inventors Melinte, et al., filed Sep. 17, 2001, and issued Mar. 2, 2004, is directed to a three solution application method for tattoo removal.

US Patent Publication No. 2010/0191216 for Indications for local transport of anaesthetic agents by electrotransport devices by inventors Keusch, et al., filed Jun. 26, 2009, and published Jul. 29, 2010, is directed to the use of an iontophoresis electrode assembly for delivery of a drug formulation. The drug formulation includes an anaesthetic and a vasoconstrictor. It is administered to a patient prior to a procedure to produce clinically acceptable depth and duration of dermal anaesthesia at the portion of skin to subject to a painful procedure or to reduce or eliminate pain. The procedure is one selected from the group consisting of venipuncture, IV cannulation, needle aspirations, body piercings, blood donations, electrolysis, tattoo removal, tattoo application, injections, dermabrasion, skin peeling, high velocity particle ablation, pace maker implantation, pace maker replacement, epidural puncture, lumbar puncture, regional nerve blocks, skin harvesting, small skin incisions, skin biopsies, circumcisions or excisions. The iontophoresis electrode assembly may also be used to reduce or temporarily eliminate neuropathic pain.

US Patent Publication No. 2007/0078434 for Indications for local transport of anaesthetic agents by electrotransport devices by inventors Keusch, et al., filed Sep. 29, 2006, and published Apr. 5, 2007, is directed to the use of an iontophoresis electrode assembly for delivery of a drug formulation. The drug formulation includes an anaesthetic and a vasoconstrictor. It is administered to a patient prior to a procedure to produce clinically acceptable depth and duration of dermal anaesthesia at the portion of skin to subject to a painful procedure or to reduce or eliminate pain. The procedure is one selected from the group consisting of venipuncture, IV cannulation, needle aspirations, body piercings, blood donations, electrolysis, tattoo removal, tattoo application, injections, dermabrasion, skin peeling, high velocity particle ablation, pace maker implantation, pace maker replacement, epidural puncture, lumbar puncture, regional nerve blocks, skin harvesting, small skin incisions, skin biopsies, circumcisions or excisions. The iontophoresis electrode assembly may also be used to reduce or temporarily eliminate neuropathic pain.

US Patent Publication No. 2018/0353324 for Thermal control of liquids for transcutaneous delivery by inventors Goldenberg, et al., filed May 30, 2018, and published Dec. 13, 2018, is directed to a device for controlling the temperature of liquids delivered to the skin during cosmetic, diagnostic, or curative procedures. Providing procedures for treating the skin, including an efficient procedure for removing pigments or tattoos, employing a liquid delivered to the skin with precisely controlled temperature and flowrate.

US Patent Publication No. 2024/0366924 for System for puncturing the epidermis with controlled fluid delivery by inventors Niven, et al., filed May 20, 2024, and published Nov. 7, 2024, is directed to an actuating device for actuating a needle cartridge and puncturing an epidermis with controlled depth. The actuating device includes a motor housed in a motor housing, an actuating rod driven by the motor and configured to actuate in a reciprocating motion, and an adjustment mechanism. The actuating rod is housed in a rod housing, the rod housing forming a device aperture configured to receive a needle cartridge and to attach to the needle cartridge. The adjustment mechanism interfaces with the rod housing and is configured to adjust a position of the rod housing relative to the motor housing while not rotating the rod housing relative to the motor housing.

US Patent Publication No. 2025/0161183 for Compositions and methods for the removal of tattoos by inventor Falkenham, filed Jan. 16, 2025, and published May 22, 2025, is directed to a method for removing a tattoo in a region of skin the method comprises administering to a least a portion of the tattoo a composition comprising an effective amount of a bisphosphonate and at least one pharmaceutically acceptable excipient to at least cause fading of the tattoo in said region.

US Patent Publication No. 2024/0216035 for Needle adaptor for plasma generating handpiece by inventors Schmieg, et al., filed Nov. 20, 2023, and published Jul. 4, 2024, is directed to methods and devices for adapting plasma generating systems for used with one or more needles, such that a plasma can be generated at the distal end or tip of the needle(s). An adaptor with an energy conductor is employed to deliver energy to the tip of the needle at a target site. A fluid, e.g., a liquid, gas or liquid/gas mixture, can also be delivered by the adaptor to the needle to provide an ionizable substance for plasma formation and/or a mobilization fluid to facilitate removal of material that has been plasma-treated. The adaptors can be used, for example, in conjunction with plasma generating handpiece to degrade and/or dislodge tattoo in particles in a tattooed region of a subject's dermis.

US Patent Publication No. 2022/0176094 for Needling device and drug applicator by inventors Tharp, et al., filed Oct. 15, 2021, and published Jun. 9, 2022, is directed to a needling device may be used for needling of a subject's skin, and a drug applicator device may be used for applying a drug to a subject's skin. For example, a needling device may be applied to a subject's skin for hair growth applications, or may be used for wrinkle, scar, or tattoo removal. A drug applicator device may be used for multiple drug application purposes such as applying a hair growth compound to the skin of a subject.

### SUMMARY OF THE INVENTION

The present invention relates to tattoo and permanent makeup removal.

It is an object of this invention to provide a new, non-laser method of removing tattoos and permanent makeup including providing simultaneous numbing and removal of the tattoo or permanent makeup through a laser-free tattoo removal pen.

In one embodiment, the present invention includes a device for laser-free tattoo removal including a head piece comprising a needle cartridge including at least one needle bundle, at least one fillable glass syringe containing tattoo removal fluid, at least one second fillable glass syringe containing numbing fluid, an internal dual syringe pump, and a Y-shaped fluid connector, wherein the internal dual syringe pump is operable to administer the tattoo removal fluid and the numbing fluid by pushing a plunger of the at least one fillable glass syringe containing tattoo removal fluid and by pushing a plunger of the at least one second fillable glass syringe containing numbing fluid, wherein the internal dual syringe pump is operable to control the flow rates of the tattoo removal fluid and the numbing fluid, such that the tattoo removal fluid and the numbing fluid are operable to be administered at the same or different flow rates, wherein the Y-shaped fluid connector is operable to connect the tattoo removal fluid and the numbing fluid, wherein the tattoo removal fluid and the numbing fluid are delivered through the head piece, and wherein the device is operable to administer the tattoo removal fluid and the numbing fluid through the head piece simultaneously.

In another embodiment, the present invention includes a method for tattoo removal including determining a first treatment area of skin of the human patient, marking a perimeter of the first treatment area of skin of the human patient with a surgical marker, abrading the first treatment area of skin with at least one needle bundle of the laser-free device by creating a plurality of small shapes with the at least one needle bundle on the first treatment area of skin, and delivering, with a head piece of the laser-free device, a tattoo removal fluid to the first treatment area of skin of the human patient while simultaneously delivering, with a head piece of the laser-free device, a numbing fluid to the first treatment area of skin of the human patient.

In yet another embodiment, the present invention includes a device for laser-free tattoo removal, including a head piece comprising a needle cartridge including at least one needle bundle, at least one fillable glass syringe containing tattoo removal fluid, at least one second fillable glass syringe containing numbing fluid, at least two stepper motors, and a touch screen, wherein the at least one fillable glass syringe containing the tattoo removal fluid is refillable, wherein the at least one fillable glass syringe containing the tattoo removal fluid includes at least one tattoo removal fluid refill hole, wherein the at least one fillable glass syringe containing the numbing fluid is refillable, wherein the at least one second fillable glass syringe containing the numbing fluid includes at least one numbing fluid refill hole, wherein a stepper motor of the at least two stepper motors is operable to administer the tattoo removal fluid, wherein a second stepper motor of the at least two stepper motors is operable to administer the numbing fluid, wherein the at least two stepper motors are operable to control the flow rates of the tattoo removal fluid and the numbing fluid, such that the tattoo removal fluid and the numbing fluid are operable to be administered at the same or different flow rates, wherein the touch screen is operable to display a depth of the at least one needle bundle, wherein the touch screen is further operable to display a speed of the device, wherein the tattoo removal fluid and the numbing fluid are delivered through the head piece, and wherein the device is operable to administer the tattoo removal fluid and the numbing fluid through the head piece simultaneously.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 illustrates a front view of a tattoo removal machine of the present invention.

FIG. 2A illustrates a front view of a tattoo removal pen of the present invention.

FIG. 2B illustrates a second front view of a tattoo removal pen of the present invention.

FIG. 2C illustrates a third front view of a tattoo removal pen of the present invention.

FIG. 2D illustrates a fourth front view of a tattoo removal pen of the present invention.

FIG. 2E illustrates an interior front view of a tattoo removal pen of the present invention.

FIG. 2F illustrates a second interior front view of a tattoo removal pen of the present invention.

FIG. 2G illustrates an additional interior front view of a tattoo removal pen of the present invention.

FIG. 2H illustrates an additional interior front view of the multiple micro machine housing and the syringe housing of the tattoo removal pen of the present invention.

FIG. 3A illustrates a removal fluid canister of the present invention.

FIG. 3B illustrates a numbing fluid canister of the present invention.

FIG. 4 illustrates a first front perspective view of a dual Y clip of the present invention.

FIG. 5 illustrates a second front perspective view of a dual Y clip of the present invention.

FIG. 6 illustrates a front orthogonal view of a dual Y clip of the present invention.

FIG. 7 illustrates an enlarged view of a port of a dual Y clip of the present invention.

FIG. 8 illustrates a back orthogonal view of a dual Y clip of the present invention.

FIG. 9 illustrates a top orthogonal view of a dual Y clip of the present invention.

FIG. 10 illustrates a back perspective view of a dual Y clip of the present invention.

FIG. 11 illustrates a side orthogonal view of a dual Y clip stem of the present invention.

FIG. 12 illustrates a tattoo removal machine, according to one embodiment of the present invention.

FIG. 13 illustrates a tattoo removal machine according to one embodiment of the present invention.

FIG. 14 illustrates a tattoo removal machine according to one embodiment of the present invention.

FIG. 15 is a schematic diagram of a system of the present invention.

### DETAILED DESCRIPTION

The present invention is generally directed to tattoo and permanent makeup removal.

In one embodiment, the present invention includes a device for laser-free tattoo removal including a head piece comprising a needle cartridge including at least one needle bundle, at least one fillable glass syringe containing tattoo removal fluid, at least one second fillable glass syringe containing numbing fluid, an internal dual syringe pump, and a Y-shaped fluid connector, wherein the internal dual syringe pump is operable to administer the tattoo removal fluid and the numbing fluid by pushing a plunger of the at least one fillable glass syringe containing tattoo removal fluid and by pushing a plunger of the at least one second fillable glass syringe containing numbing fluid, wherein the internal dual syringe pump is operable to control the flow rates of the tattoo removal fluid and the numbing fluid, such that the tattoo removal fluid and the numbing fluid are operable to be administered at the same or different flow rates, wherein the tattoo removal fluid and the numbing fluid are delivered through the head piece, wherein the device is operable to administer the tattoo removal fluid and the numbing fluid through the head piece simultaneously, wherein the device is operable to abrade a first treatment area of skin by creating by plurality of small shapes with the at least one needle bundle on the first treatment area of skin, wherein the device further includes at least one screen, wherein the at least one screen is a touch screen, wherein the screen is operable to display the depth of the at least one needle bundle when the device is in use, wherein the screen is further operable to display the device speed when the device is in use, wherein the at least one fillable glass syringe containing the tattoo removal fluid is refillable, wherein the at least one fillable glass syringe containing the tattoo removal fluid includes at least one tattoo removal fluid refill hole, wherein the at least one second fillable glass syringe containing the numbing fluid is refillable, wherein the at least one second fillable glass syringe containing the numbing fluid includes at least one numbing fluid refill hole, wherein the device is operable to wirelessly connect to an external device, wherein the device includes software, wherein the software is operable to be updated through a wireless update, wherein the device is operable to measure the amount of tattoo removal fluid and/or numbing fluid, wherein the device is operable to produce an automatic alert if the amount of tattoo removal fluid and/or the amount of the numbing fluid fall below a set threshold level, and wherein the software is operable to automatically shutoff the device.

In another embodiment, the present invention includes a method for tattoo removal including determining a first treatment area of skin of the human patient, marking a perimeter of the first treatment area of skin of the human patient with a surgical marker, abrading the first treatment area of skin with at least one needle bundle of the laser-free device by creating a plurality of small shapes with the at least one needle bundle on the first treatment area of skin, delivering, with a head piece of the laser-free device, a tattoo removal fluid to the first treatment area of skin of the human patient while simultaneously delivering, with the head piece of the laser-free device, a numbing fluid to the first treatment area of skin of the human patient, wherein the method does not include templating the first treatment area, further comprising spacing at least one small shape of the plurality of small shapes at least 4 mm apart from a second at least one small shape of the plurality of small shapes, further comprising administering the tattoo removal fluid and the numbing fluid by an internal dual syringe pump of the laser-free device, further comprising pushing, by the internal dual syringe pump, a plunger of a fillable glass syringe containing tattoo removal fluid, and pushing, by the internal dual syringe pump, a plunger of a fillable glass syringe containing numbing fluid, further comprising controlling, by the internal dual syringe pump, the flow rates of the tattoo removal fluid and the numbing fluid, such that the tattoo removal fluid and the numbing fluid are operable to be administered at the same or different flow rates, and further comprising determining a depth of the needle bundle required for removing the tattoo.

In yet another embodiment, the present invention includes a device for laser-free tattoo removal, including a needle cartridge including at least one needle bundle, at least one fillable glass syringe containing tattoo removal fluid, at least one second fillable glass syringe containing numbing fluid, at least two stepper motors, and a touch screen, wherein the at least one fillable glass syringe containing the tattoo removal fluid is refillable, wherein the at least one fillable glass syringe containing the tattoo removal fluid includes at least one tattoo removal fluid refill hole, wherein the at least one fillable glass syringe containing the numbing fluid is refillable, wherein the at least one second fillable glass syringe containing the numbing fluid includes at least one numbing fluid refill hole, wherein a stepper motor of the at least two stepper motors is operable to administer the tattoo removal fluid, wherein a second stepper motor of the at least two stepper motors is operable to administer the numbing fluid, wherein the at least two stepper motors are operable to control the flow rates of the tattoo removal fluid and the numbing fluid, such that the tattoo removal fluid and the numbing fluid are operable to be administered at the same or different flow rates, wherein the touch screen is operable to display a depth of the at least one needle bundle, wherein the touch screen is further operable to display a speed of the device, wherein the tattoo removal fluid and the numbing fluid are delivered through the head piece, wherein the device is operable to administer the tattoo removal fluid and the numbing fluid through the head piece simultaneously, wherein the device is operable to abrade a first treatment area of skin by creating by plurality of small shapes with the at least one needle bundle on the first treatment area of skin, wherein the device includes a Y-shaped fluid connector and a mixing agitator, wherein the Y-shaped fluid connector is operable to connect the tattoo removal fluid and the numbing fluid, wherein the mixing agitator is operable to mix the tattoo removal fluid and the numbing fluid together, wherein the device is operable to measure the amount of tattoo removal fluid and/or numbing fluid, wherein the device is operable to produce an automatic alert if the amount of tattoo removal fluid and/or the amount of the numbing fluid fall below a set threshold level, and wherein the at least one needle bundle includes at least five needles arranged in a star-shaped pattern.

None of the prior art discloses a non-laser tattoo removal system that includes a laser-free device that simultaneously delivers both a regulated numbing fluid and a proprietary removal fluid, used during the removal procedure of tattoos or permanent makeup.

A tattoo is a form of body modification including a permanent mark or design made on the skin with tattoo ink, pigment, and/or dye. Generally, a tattoo artist utilizes a handheld machine which includes needles that insert droplets of ink into the top layer of the skin to apply a tattoo.

Individuals receive tattoos under many different circumstances, including, but not limited to, personal and creative choice, identification and trauma circumstances, medical circumstances, religious and cultural ritual, and/or cosmetic circumstances.

Permanent makeup, also known as micropigmentation, permanent cosmetics, or cosmetic tattooing, is a cosmetic procedure utilizing tattooing as a means to implant pigment into the upper layers of the skin to mimic makeup and enhance facial features. Common permanent makeup procedures involve enhancements to the eyebrows, lips, freckles, and/or hair/scalp. Common permanent makeup applications include microblading eyebrows, permanent eyeliner, lip blushing, eyebrow shading, lip liner color, and/or full lip color.

Tattoo or permanent makeup removal is a procedure performed to remove unwanted tattoos or permanent makeup. Individuals pursue tattoo removal for many different reasons, such as the aged appearance of the tattoo, fading or blurring of the tattoo, poor design, social stigma, life changes, changed personal appearance choices, and/or the tattoo carries a negative association such as a traumatic experience for an individual, such as human trafficking.

Common techniques for tattoo removal include laser removal, surgical excision, dermabrasion, chemical peels, saline removal, and removal creams/ointments. Laser removal is the most common and efficacious method of tattoo removal currently in the art. Laser tattoo removal works by focusing laser beams of light onto a client's skin, heating up the ink particles of a tattoo to break them into smaller pieces. By breaking the ink into smaller pieces, the human immune system uses white blood cells to disperse the ink particles. Generally, laser tattoo removal requires several sessions to complete the removal, averaging six to ten sessions per client. However, larger tattoos often take ten to twenty sessions and generally all sessions need to be scheduled at least six to eight weeks apart to give the skin time to heal between treatments. This demonstrates the extremely long amount of time a client must wait for completion of the tattoo removal, not to mention how many times a client must have the same area of skin repeatedly invaded in order to have a tattoo removed. Hence there is a need for a method of tattoo removal that is quicker, faster, and less traumatic to a client's skin than laser tattoo removal.

Dermabrasion involves essentially sanding away the top layer of skin with an abrasive wheel or brush, allowing the tattoo ink to hopefully leach out of the skin. Surgical excision involves cutting out the skin that the tattoo is on. Chemical peels involve acids applied to the skin to remove the outer layers of the skin, and hopefully, the tattoo ink along with the outer layer of skin. These techniques do not technically remove pigment, but merely remove only the skin the pigment/ink is under. These techniques aim to stimulate an immune response to lighten or remove the ink pigments, which ultimately result in permanent damage to the skin. As such, these methods have highly variable results and likely do not fully remove all of the tattoo ink from an individual's tattoo. Thus, there is a need for a more effective method of tattoo removal.

Additionally, there have been no FDA approved tattoo removal ointments or creams, including any do-it-yourself tattoo removal kits. Generally, a tattoo removal cream includes bleaching creams, peeling agents/acids, and/or retinol, which exfoliate the skin. However, it is unlikely for any of these ingredients to actually reach the tattoo ink pigments in the deeper skin layers, and as such, are not very effective. Thus, there is a need for a more effective method of tattoo removal.

In addition to not being very effective or time efficient, these known methods can cause serious damage to the skin, coupled with leaving a client's skin uncared for after the procedures are over. These common tattoo removal techniques currently known in the art, include excision, dermabrasion, enzymatic salabrasion, and laser removal. Each of these known methods have many risks and side effects. However, all of the listed removal methods, have risks that can cause damage to the skin, including scarring, infection, skin discoloration, inflammation, irritation, blistering, crusting, bruising, bleeding, redness, soreness, swelling, discomfort, intense pain and/or allergic reactions. Laser removal in particular can also cause blistering, and frosting, as well as two other major side effects that will occur after the procedure, which are undetectable by the client or the technician. The first of these side effects includes the client's tattoo turning permanently green after going under a 900° F. laser, commonly used in laser tattoo removal, which reveals there is metal in the tattoo ink, and laser removal will only give that result as it cannot fully remove the metal. The second undetectable side effect that may occur after laser removal includes hypertrophic and keloid scarring that is not caused by invasion of the skin alone, but also caused by a 900° F. laser hitting ink that has undetected acrylic (plastic), an ingredient within some tattoo inks. Although any invasion to the skin could cause a number of these side effects, the system of the present invention advantageously demonstrates how to avoid and prevent these side effects.

Tattoo removal is often extremely difficult, long, physically painful, and expensive for a client. As previously mentioned, tattoo removal methods generally require numerous procedures to fully remove a tattoo, which frequently require months or years for full removal, resulting in greater financial costs, pain, and side effects to the client. Moreover, in some instances a tattoo cannot be fully removed and/or is never fully gone from the client's skin, even after many treatments. The financial burden on clients, which clients endure session after session, has also prompted the need for new methods of tattoo removal. Thus, what is needed is a new, non-laser method of tattoo removal that is more effective, comfortable and tolerable for the client, better for the client's body, as well as quicker and less of a financial burden.

Thus, the system of the present invention includes a proprietary package with a laser-free tattoo removal system that is used to remove unwanted ink quicker, effectively, cost efficiently, with little to no pain, and caters to the integrity of the skin, which results in minimal scarring and changes in pigmentation. The system of the present invention extracts ink out of the client's body, causing no damage to lymph nodes, which lasers commonly cause when used to disperse ink into the body causing severe health issues.

Referring now to the drawings in general, the illustrations are for the purpose of describing one or more preferred embodiments of the invention and are not intended to limit the invention thereto.

In an alternative embodiment, the system of the present invention includes a tattoo removal machine, controlling software, documenting procedure information and connecting part of the of the tattoo removal machine, a wired and BLUETOOTH capable tattoo removal pen, a tattoo removal pen stand, a proprietary needle cartridge, a proprietary dual Y clip, a proprietary numbing solution, a proprietary removal solution, a proprietary dual syringe infusion pump, a dual pump auto bevel, the software of the tattoo removal machine, a tattoo removal pen, a tattoo removal pen stand, a needle cartridge, a dual Y clip, a numbing solution, a removal solution, syringe IV lines, and medical-grade aftercare products.

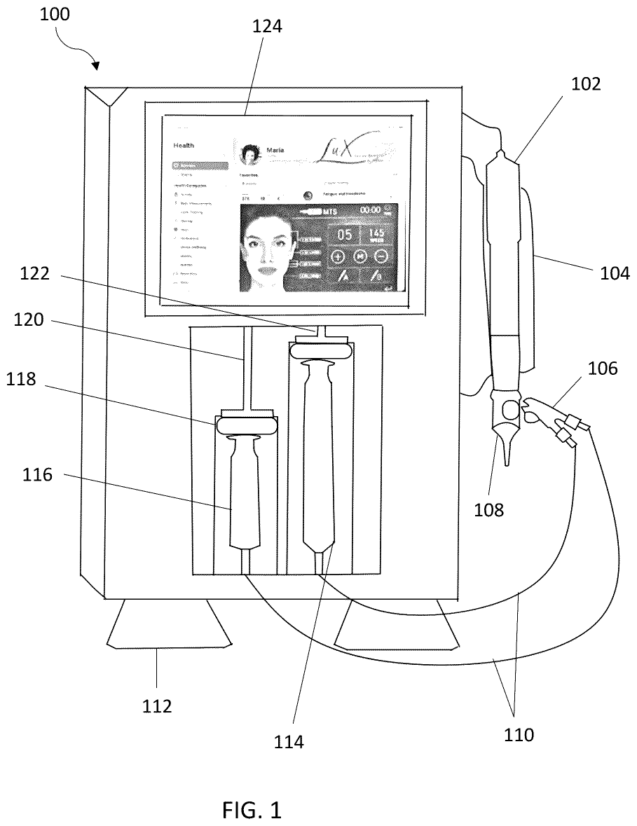

FIG. 1 illustrates a front view of a tattoo removal machine 100 of an alternative embodiment of the present invention, including a tattoo removal pen 102, a tattoo removal pen stand 104, a dual Y clip 106, a head piece 108 containing a needle cartridge, syringe intravenous (IV) lines 110, legs 112, a fillable and refillable glass syringe containing removal fluid 114, a fillable and refillable glass syringe containing numbing fluid 116, a dual syringe pump 118, a pump auto bevel for the numbing fluid 120, a pump auto bevel for the removal fluid 122, and a touch screen 124. In one embodiment, the tattoo removal machine 100 is operable to be used during a tattoo and/or permanent makeup removal procedure. The present invention is discussed primarily with respect to tattoo removal herein, but one of ordinary skill in the art recognizes that embodiments which discuss tattoo removal are also applicable for permanent makeup removal.

In one embodiment, the tattoo removal machine 100 is approximately 10 inches wide, approximately 14 inches long and approximately 2 inches deep. However, one of ordinary skill in the art will understand that the width, length, and depth of the tattoo removal machine 100 are not intended to be limiting according to the present invention.

In one embodiment, the tattoo removal pen 102 is used by a technician to remove a tattoo and/or permanent makeup. In one embodiment, the tattoo removal pen 102 is operable to wirelessly connect to the tattoo removal machine 100 via BLUETOOTH technology, providing for communication between the tattoo removal pen 102 and the tattoo removal machine 100. In one embodiment, the tattoo removal pen 102 is operable to wirelessly connect to the tattoo removal machine 100 via a plurality of wireless technologies, including Wi-Fi Direct, near-field communication, ZIGBEE, Z-WAVE, 6LOWPAN, XENDER, and/or local area network technology. In one embodiment, the tattoo removal pen 102 is operable to connect to the tattoo removal machine 100 via a wired connection.

In one embodiment, commands are sent from the tattoo removal machine 100 to the tattoo removal pen 102 during a procedure to modify or adjust the depth of the tattoo removal pen 102 on the client's skin. In one embodiment, commands are sent from the tattoo removal machine 100 to the tattoo removal pen 102 during a procedure to modify or adjust the speed of the tattoo removal pen 102 on the client's skin. In one embodiment, the tattoo removal pen 102 is only operable for tattoo and/or permanent makeup removal procedures.

In one embodiment, the tattoo removal pen 102 is operable to remove tattoos and/or permanent makeup according to the method of tattoo removal the present invention. In one embodiment, the method of tattoo removal provides for the removal of tattoos and/or permanent makeup by abrading the skin via small unique shaped wounds over the tattoo or permanent makeup area. Advantageously, the method does not use a template. In one embodiment, the method of tattoo removal of the present invention includes determining the desired treatment area of skin to be worked on, including a discussion between a technician and a client. In one embodiment, the method of tattoo removal includes cleaning the treatment area before the procedure. In one embodiment, the method includes photographing the treatment area before the procedure and uploading the photos to a patient's chart. In one embodiment, the method of tattoo removal includes using a surgical marker to mark off a treatment area with lines marking the perimeter, wherein the perimeter is no larger than two four-by-four-inch areas, only on the left or right side of the body. In one embodiment, the method of tattoo removal includes determining the depth of the needle required for the treatment, depending on the density of the skin. In one embodiment, the method of tattoo removal includes considering the age of the client to determine the specific technique of needle movement during abrading the skin to protect the skin. In one embodiment, the method of tattoo removal advantageously includes creating small, unique shapes on the existing ink, ensuring a space of at least 4 mm in between each treatment spot, wherein these shapes are not more than 4 mm high or 8 mm long, wherein these shapes are never completely wrapped around a limb or finger during removal. In one embodiment, each shape has at least 4 mm between each shape to ensure proper healing. In one embodiment, the shapes are designed to cover the designated area of skin chosen for the session of ink removal. In one embodiment, the shapes have a depth of 1 mm to 1.5 mm into the epidermis layer of the skin, depending on the area to be treated. In one embodiment, the method of tattoo removal of the present invention includes the tattoo removal pen 102 which includes a least one bundle of multiple needles operable to simultaneously create the small, unique shapes on the existing ink. In one embodiment, the bundle of needles is operable to abrade the skin by oscillating along the marked treatment area. In one embodiment, the tattoo removal fluid of the present invention is operable to soften the epidermis layer of skin, allowing the oscillating needle bundle to more efficiently create a small surface wound, which allows the ink to draw to the surface of the skin. The small surface wound created by the bundle of needles creates an opening for the tattoo ink pigment to rise to the surface of the skin when exposed to oxygen, collecting on the underside of a scab which forms over the small surface wound. In one embodiment, the method of tattoo removal of the present invention includes five needles arranged in a star pattern. In one embodiment, the method of tattoo removal of the present invention includes laser-light mapping of the tattooed skin, wherein the tattoo removal pen 102 includes a laser-light mapping device.

In one embodiment, the tattoo removal pen stand 104 is operable to hold the tattoo removal pen 102 when the pen is not in use, further operable to position the tattoo removal pen 102 facing down, advantageously prohibiting the fluid from flowing back into the tattoo removal pen 102 and ruining the tattoo removal pen 102. In one embodiment, the tattoo removal pen stand 104 is attached to and extends outwardly from a side, back or front of the tattoo removal machine 100 of the present invention. In one embodiment, the tattoo removal pen stand 104 is separate and not attached to the tattoo removal machine 100 of the present invention. In one embodiment, the tattoo removal pen stand 104 is made of plastic. In one embodiment, the tattoo removal pen stand 104 is made of silicone.

In one embodiment, the syringe IV lines 110 transport the removal fluid contained in a fillable and refillable glass syringe 114 and the numbing fluid contained in a fillable and refillable glass syringe 116 to the Y clip 106 to continuously drip down the head piece 108 containing a needle cartridge connected to the tattoo removal pen 102. In one embodiment, there is one IV line 110 for the removal fluid and one IV line 110 for the numbing fluid. In one embodiment, there is one IV line 110 for both the removal fluid and the numbing fluid to be combined. In one embodiment, there are more than two IV lines 110.

In one embodiment, the legs 112 of the tattoo removal machine 100 are two separate vertical standing legs. In one embodiment, the legs 112 of the tattoo removal machine 100 are two connected vertical standing legs. In one embodiment, a leg 112 of the tattoo removal machine 100 is one single vertical standing leg. In one embodiment, the tattoo removal machine 100 is operable to stand on one leg 112. In one embodiment, the tattoo removal machine 100 is operable to stand on two legs 112. In one embodiment, the tattoo removal machine 100 is operable to stand on a plurality of legs 112. In one embodiment, the tattoo removal machine 100 is operable to stand on more than two legs 112. In one embodiment, the tattoo removal machine 100 is operable to stand without any legs 112. In one embodiment, the legs 112 of the tattoo removal machine 100 are operable to be removed from the tattoo removal machine 100 for cleaning purposes. In one embodiment, the legs 112 of the tattoo removal machine 100 are permanent and cannot be removed from the tattoo removal machine 100. In one embodiment, the legs 112 of the tattoo removal machine 100 are made of plastic. In one embodiment, the legs 112 of the tattoo removal machine 100 are made of silicone. In one embodiment, the legs 112 of the tattoo removal machine 100 are made of metal. In one embodiment, the legs 112 of the tattoo removal machine 100 are made of stainless steel. In one embodiment, the legs 112 of the tattoo removal machine 100 are operable to be formed in a plurality of different shapes.

In an alternative embodiment, the removal fluid is prepackaged in a prefilled syringe, which is operable to be inserted into the tattoo removal machine 100 and attached to the syringe IV lines 110. In one embodiment, the syringe is not pre-filled. In one embodiment, the syringe is fillable and refillable. In a preferred embodiment, the syringe is made of glass. In one embodiment, the fillable and refillable glass syringe containing removal fluid 114 includes 4 mL of removal fluid.

However, one of ordinary skill in the art will understand that the amount of removal fluid in the syringe is not intended to be limiting according to the present invention. In one embodiment, the removal fluid is operable to prepare the skin during the tattoo removal process for removal by softening the epidermis layer of the skin, advantageously permitting the needles to not abrade dry skin, thus catering to the integrity of the skin. In one embodiment, the removal fluid is operable to remove the tattoo ink from a client's skin during the procedure. In one embodiment, the removal fluid is operable to assist in collecting the ink during the tattoo removal process. In one embodiment, the removal fluid is operable to include healing components within the formula. In one embodiment, the removal fluid is operable to be administered during the procedure via the Y clip 106 and the head piece 108 containing a needle cartridge of the present invention. In one embodiment, the removal fluid is operable to be administered during the procedure via the head piece 108 containing a needle cartridge of the present invention. The tattoo removal fluid is operable to include acids such as lactic acid, butyric acid, citric acid, trichloroacetic acid, glycolic acid, hydrogen peroxide, and/or one or more antiseptics. The tattoo removal fluid is further operable to include water and salt. In one embodiment, the tattoo removal fluid is operable to include hydroquinone. In one embodiment, the tattoo removal fluid is further operable to include aloe and/or one or more skin healing ingredients.

In an alternative embodiment, the numbing fluid is prepackaged in a prefilled syringe, which is operable to be inserted into the tattoo removal machine 100 and attached to the syringe IV lines 110. In a preferred embodiment, the syringe is not pre-filled and operable to be filled before a procedure and refilled during and/or after a procedure. In one embodiment, the syringe is fillable and refillable. In a preferred embodiment, the syringe is made of glass. In one embodiment, the numbing fluid syringe includes 4 mL of numbing fluid. However, one of ordinary skill in the art will understand that the amount of numbing fluid in the syringe is not intended to be limiting according to the present invention. In one embodiment, the numbing fluid is operable to be administered on broken and/or unbroken skin. In one embodiment, the numbing fluid is operable to be administered during the procedure via the Y clip 106 and the head piece 108 containing a needle cartridge of the present invention. In one embodiment, the numbing fluid is operable to be administered before the procedure via the head piece 108 containing a needle cartridge of the present invention. In one embodiment, the numbing fluid is operable to be administered after the procedure via the head piece 108 containing a needle cartridge of the present invention. The numbing fluid is operable to include lidocaine in one embodiment.

In one embodiment, the dual syringe pump 118 is operable to administer the removal fluid contained in a fillable and refillable glass syringe 114 and the numbing fluid contained in a fillable and refillable glass syringe 116, through the IV lines 110 and to the Y clip 106 to produce drips of each fluid through the needle cartridge of the head piece 108 and onto the skin of a client during a procedure. In one embodiment, the dual syringe pump 118 is operable to administer only the removal fluid through the IV lines 110 during a procedure. In one embodiment, the dual syringe pump 118 is operable to administer only the numbing fluid through the IV lines 110 during a procedure. In one embodiment, the dual syringe pump 118 is operable to administer the removal fluid and the numbing fluid at the same rates, simultaneously. In one embodiment, the dual syringe pump 118 is operable to administer the removal fluid and the numbing fluid simultaneously, but at different rates. In one embodiment, the tattoo removal machine includes dual syringe pump controls operable to control the flow of the removal fluid and/or the numbing fluid.

In one embodiment, the pump auto bevel for the numbing fluid 120 is operable to function simultaneously with the pump auto bevel for the removal fluid 122, along with the dual syringe pump 118 to administer the removal fluid and the numbing fluid through the IV lines 110 during a procedure. In one embodiment, the pump auto bevels 120 and 122 are operable to ensure level distribution of the removal fluid and/or the numbing fluid. In one embodiment, the pump auto bevel for the numbing fluid 120 and the pump auto bevel for the numbing fluid 120 are operable to operate at different flow rates. In one embodiment, the pump auto bevel for the numbing fluid 120 is operable to be turned off while the pump auto bevel for the removal fluid 122 is still operating. In one embodiment, the pump auto bevel for the removal fluid 122 is operable to be turned off while the pump auto bevel for the numbing fluid 120 is still operating.

In one embodiment, the administration of the numbing fluid and/or the removal fluid is operable to be manually shut off by a technician. In one embodiment, the administration of the numbing fluid and/or the removal fluid is operable to be automatically shut off by the software of the tattoo removal machine 100.

In an alternative embodiment, the dual Y clip 106 is operable to attach onto the end of head piece 108 of the tattoo removal pen 102. In one embodiment, the dual Y clip 106 is operable to connect to at least two syringe IV lines 110. In one embodiment, the dual Y clip 106 is operable to connect at least two syringe IV lines 110 to the head piece 108 of the tattoo removal pen 102. In one embodiment, the dual Y clip 106 is operable to connect to both the syringe containing the removal fluid and the syringe containing the numbing fluid IV lines 110 to the head piece 108 of the tattoo removal pen 102. In one embodiment, the dual Y clip 106 is operable to provide for both the removal fluid and the numbing fluid to flow through the single port of the head piece 108 containing a needle cartridge, providing simultaneous numbing and removal of the tattoo or permanent makeup.

In one embodiment, the dual Y clip 106 is made of plastic. In one embodiment, the dual Y clip 106 is made of silicone.

In a preferred embodiment, the system of the present invention includes a laser-free tattoo removal pen 102.

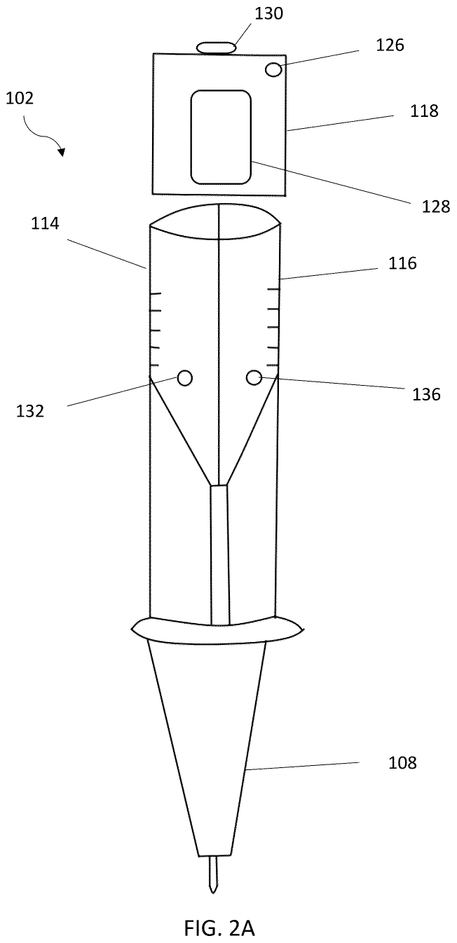

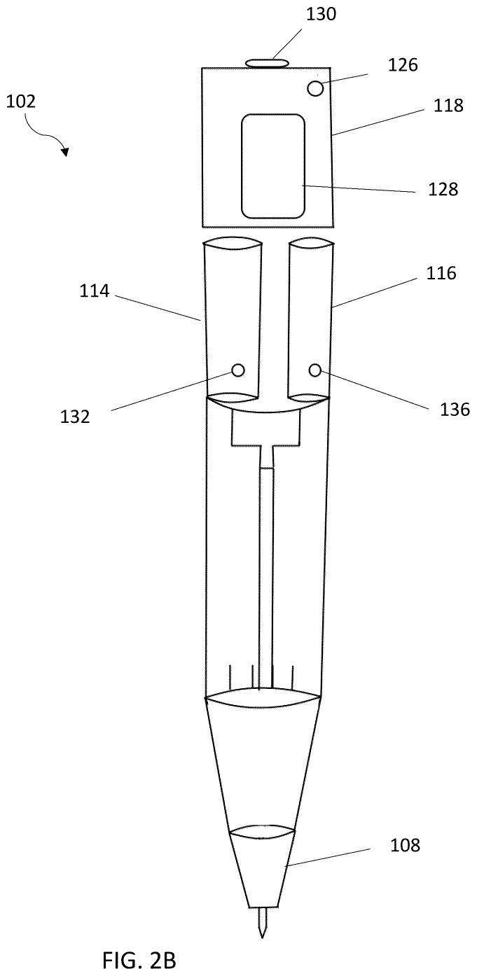

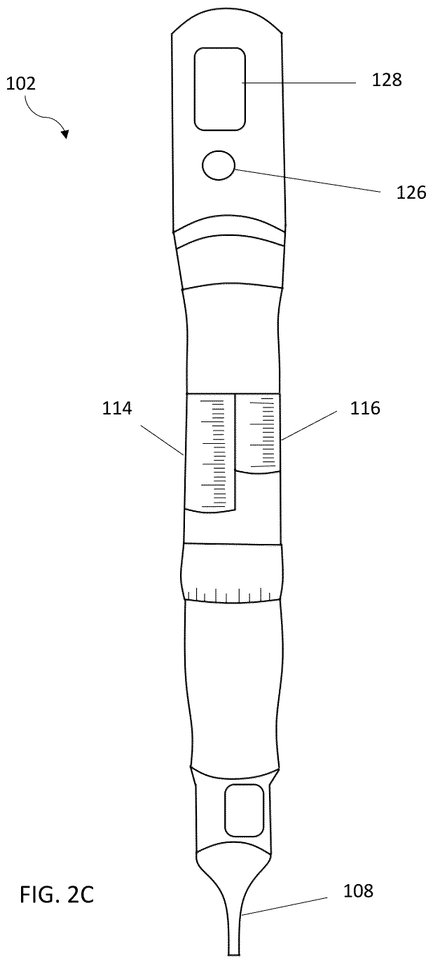

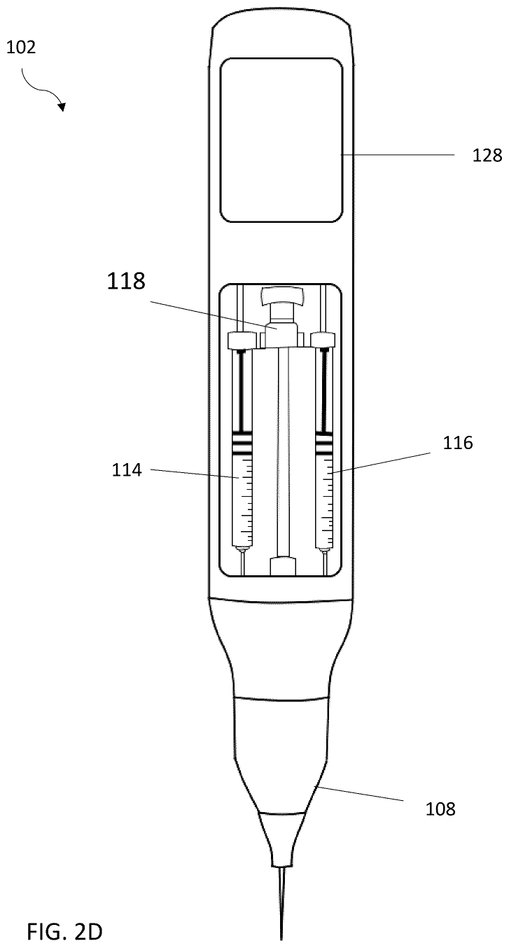

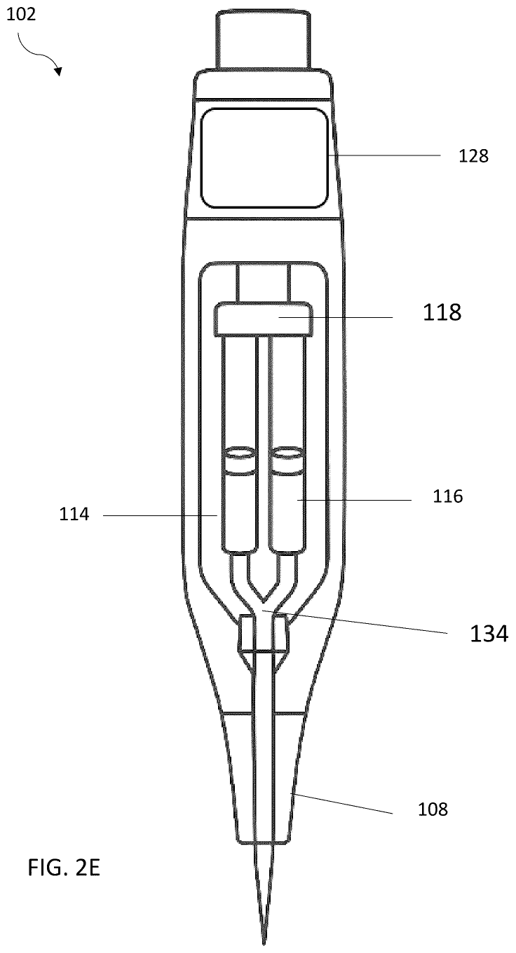

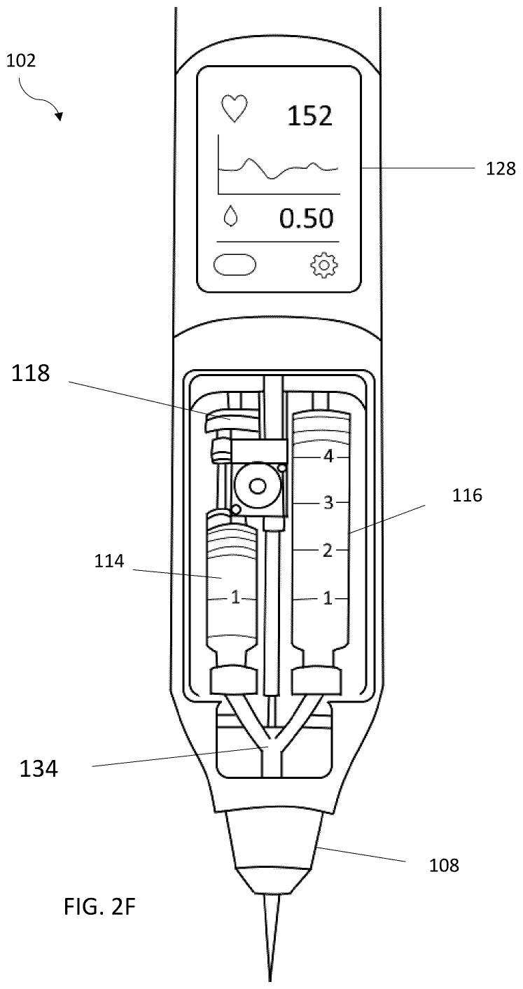

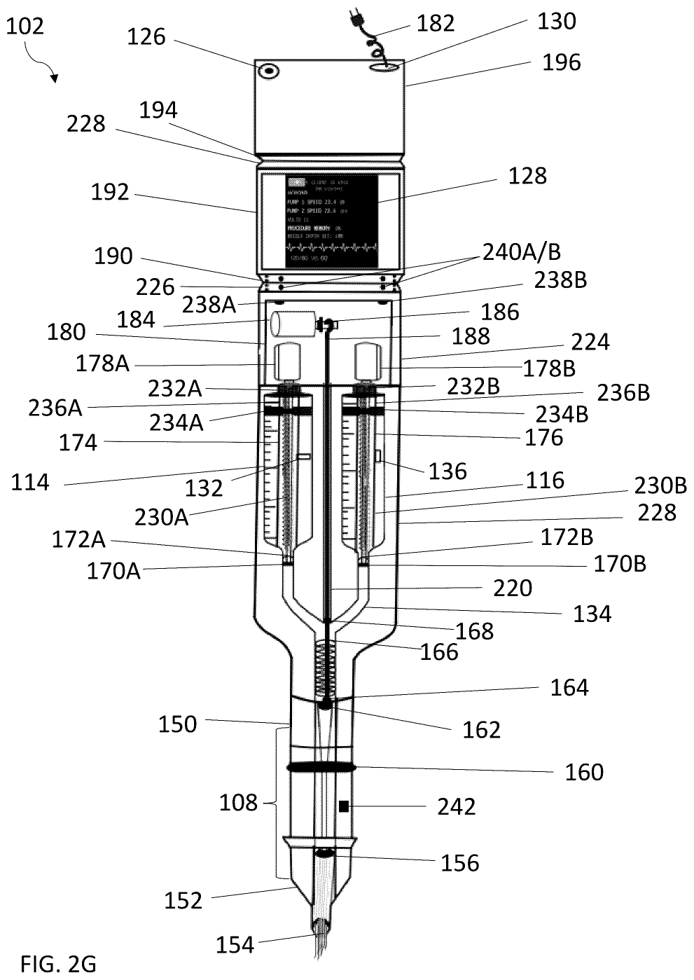

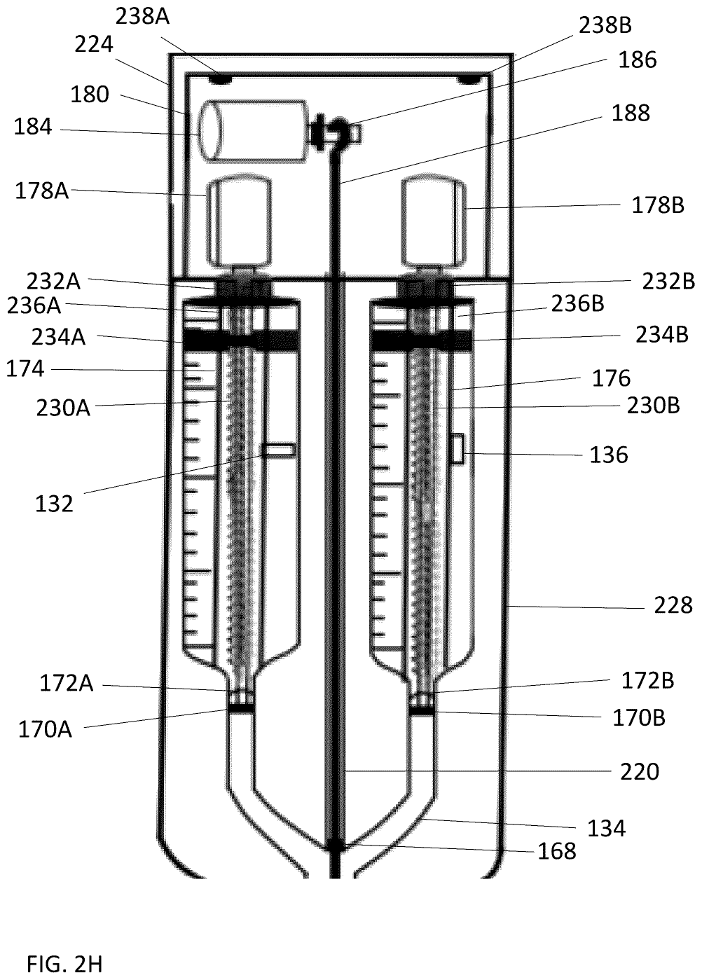

FIG. 2A illustrates a front view of a preferred embodiment of a tattoo removal pen of the present invention, generally described as 102, including a head piece 108 containing a needle cartridge with at least one needle bundle, a fillable and refillable glass syringe containing removal fluid 114, a fillable and refillable glass syringe containing numbing fluid 116, an internal dual syringe infusion pump 118, an on/off button 126, touch screen 128, a charging port 130, at least one tattoo removal fluid refill hole 132 and at least one numbing fluid refill hole 136. In one embodiment, the needle cartridge includes a needle bundle, including at least five needles. In one embodiment, the needle cartridge includes a needle bundle, including a range of five to seven needles.

In one embodiment, the internal dual syringe infusion pump 118 is operable to administer both the removal fluid and the numbing fluid by pushing the plunger of both syringes, including the syringe containing the tattoo removal fluid and the syringe containing the numbing fluid. In one embodiment, the internal dual syringe infusion pump 118 is operable to administer only the tattoo removal fluid. In one embodiment, the internal dual syringe infusion pump 118 is operable to administer only the numbing fluid. In one embodiment, the internal dual syringe infusion pump 118 is operable to administer the tattoo removal fluid and/or numbing fluid at any time during the procedure. In one embodiment, the internal dual syringe infusion pump 118 is operable to administer the tattoo removal fluid and/or numbing fluid at set intervals.

In an alternative embodiment, the tattoo removal pen 102 includes at least one removal fluid refill hole 132 and at least one numbing fluid refill hole 136. In one embodiment, the at least one removal fluid refill hole 132 and at least one numbing fluid refill hole 136 include a lock and key connector to connect to an external container of removal fluid and numbing fluid, respectfully, wherein the removal fluid contained in the a fillable and refillable glass syringe 114 and numbing fluid contained in a fillable and refillable glass syringe 116 within the tattoo removal pen 102 are operable to be refilled. In one embodiment, the at least one removal fluid refill hole 132 and at least one numbing fluid refill hole 136 are on the backside of the tattoo removal pen 102. In one embodiment, the at least one removal fluid refill hole 132 is horizontal to the syringe. In one embodiment, the at least one numbing fluid refill hole 136 is vertical to the syringe. In one embodiment, the at least one removal fluid refill hole 132 and at least one numbing fluid refill hole 136 are operable to be a circle, square, rectangle, or triangle shaped holes. However, one of ordinary skill in the art will understand that the shapes of the at least one removal fluid refill hole 132 and at least one numbing fluid refill hole 136 are not intended to be limiting according to the present invention.

In one embodiment, the tattoo removal pen 102 includes a syringe housing which contains the removal fluid syringe 114 and the numbing fluid syringe 116.

In one embodiment, the fillable and refillable glass syringe containing removal fluid 114 is operable to be inserted into the tattoo removal pen 102. In one embodiment, the fillable and refillable glass syringe containing numbing fluid 116 is operable to be inserted into the tattoo removal pen 102. In one embodiment, the fillable and refillable glass syringe containing removal fluid 114 is operable to be taken out/removed from the tattoo removal pen 102. In one embodiment, the fillable and refillable glass syringe containing numbing fluid 116 is operable to be taken out/removed from the tattoo removal pen 102. In one embodiment, the fillable and refillable glass syringe containing removal fluid 114 and/or the fillable and refillable glass syringe containing numbing fluid 116 are operable to be switched out of the tattoo removal pen 102 on an as-needed basis, advantageously providing for fast and seamless loading and reloading during a tattoo removal procedure.

In one embodiment, the fillable and refillable glass syringe containing removal fluid 114 and the fillable and refillable glass syringe containing numbing fluid 116 include a connector, wherein the connector is operable to connect and lock into the tattoo removal pen 102.

In one embodiment, the removal fluid refill is contained in a canister and/or a container, wherein the canister and/or container includes a connector on the top of the canister and/or container, wherein the connector is operable to connect and lock into the tattoo removal pen 102. In one embodiment, the numbing fluid refill is contained in a canister and/or a container, wherein the canister and/or container includes a connector on the top of the canister and/or container, wherein the connector is operable to connect and lock into the tattoo removal pen 102.

In one embodiment, the syringe, canister, or container containing the removal fluid refill or the numbing fluid refill are sterile, tamper evident, prevent exposure to air, blood borne pathogens, and/or prevent cross-contamination.

In one embodiment, the fillable and refillable glass syringe containing removal fluid 114 and the fillable and refillable glass syringe containing numbing fluid 116 are operable to include at least one removal fluid refill hole 132 and at least one numbing fluid refill hole 136, permitting the syringes to be filled and re-filled with removal and/or numbing fluid before, during, or after a procedure, as needed. In one embodiment, the tattoo removal pen 102 is operable to control the flow of the removal fluid contained in a fillable and refillable glass syringe 114 and/or the numbing fluid contained in a fillable and refillable glass syringe 116. In one embodiment, the tattoo removal pen 102 is operable to adjust the flow of the removal fluid and/or the numbing fluid at the same or different flow rates. In one embodiment, both the removal fluid and the numbing fluid flow through the head piece 108 containing a needle cartridge of the tattoo removal pen 102 simultaneously. The simultaneous delivery of the tattoo removal fluid and the numbing fluid provide for simultaneous numbing and comfort of the patient, while removing the ink from the tattoo. In one embodiment, the tattoo removal pen 102 is operable to start, stop, and/or adjust the flow of the tattoo removal fluid and the numbing fluid during a procedure.

In one embodiment, the head piece 108 containing a needle cartridge is for one-time use and is disposable. In one embodiment, the head piece 108 containing a needle cartridge is sterile. In one embodiment, the head piece 108 containing a needle cartridge connects to and locks into the end of the tattoo removal pen 102 via a lock and key mechanism. In one embodiment, the head piece 108 containing a needle cartridge includes a light to aid a technician in precise visual accuracy of the desired wound shapes to be created. In one embodiment, the head piece 108 includes at least one sensor, wherein the at least one sensor is operable to sense proper positioning of the tattoo removal pen 102 on the skin of a patient. In one embodiment, the head piece 108 is operable to rotate and click into six preset needle depths, including, but not limited to 0.05 mm, 1 mm, 1.2 mm, 1.3 mm, 1.4 mm, and 1.5 mm.

In one embodiment, the needle cartridge includes a bundle of needles. In one embodiment, the needle cartridge includes a plurality of bundles needles. In one embodiment, the needle cartridge of the head piece 108 includes at least one bundle of needles. In one embodiment, the bundle of needles is a bundle of seven bugpin round linear needles. In one embodiment, the bundle of needles includes five nano needles arranged in a star-shaped pattern, forming a circular needle tip. In one embodiment, the bundle of needles is operable to penetrate human skin at less than or equal to 1.5 mm in depth.

In one embodiment, the dual syringe pump 118 is operable to administer the removal fluid contained in a refillable glass syringe 114 and the numbing fluid contained in a fillable and refillable glass syringe by pushing the plunger of each syringe.

In a preferred embodiment, the tattoo removal pen 102 is wireless. In one embodiment, the tattoo removal pen 102 is operable to wirelessly connect to an external tablet 100 via BLUETOOTH technology, providing for communication between the tattoo removal pen 102 and the tablet, wherein the tablet includes a program for controlling the tattoo removal pen 102. In one embodiment, the tattoo removal pen 102 is operable to wirelessly connect to the tablet via a plurality of wireless technologies, including Wi-Fi Direct, near-field communication, ZIGBEE, Z-WAVE, 6LOWPAN, XENDER, and/or local area network technology. In one embodiment, the tattoo removal pen 102 is operable to connect to the tablet via a wired connection. In one embodiment, the tattoo removal pen 102 is operable to wirelessly connect via BLUETOOTH technology to any compatible device.

In one embodiment, the tattoo removal pen 102 is operable to include a tattoo removal pen holder, wherein the tattoo removal pen 102 is operable to be placed and rest on the tattoo removal pen holder, thereby ensuring the removal fluid and numbing fluid are positioned downward away from a motor of the tattoo removal pen 102.

FIG. 2B illustrates a second front view of a tattoo removal pen of the present invention, generally described as 102, including a head piece 108 containing a needle cartridge with at least one needle bundle, a fillable and refillable glass syringe containing removal fluid 114, a fillable and refillable glass syringe containing numbing fluid 116, a dual syringe pump 118, an on/off button 126, a touch screen 128, charging port 130, and at least one removal fluid refill hole 132 and at least one numbing fluid refill hole 136.

FIG. 2C illustrates a third front view of a tattoo removal pen of the present invention, generally described as 102, including a head piece 108 containing a needle cartridge with at least one needle bundle, a fillable and refillable glass syringe containing removal fluid 114, a fillable and refillable glass syringe containing numbing fluid 116, an on/off button 126, and a touch screen 128.

FIG. 2D illustrates a fourth front view of a tattoo removal pen of the present invention, generally described as 102, including a head piece 108 containing a needle cartridge with at least one needle bundle, a fillable and refillable glass syringe containing removal fluid 114, a fillable and refillable glass syringe containing numbing fluid 116, a dual syringe pump 118, and a touch screen 128.

FIG. 2E illustrates an interior front view of a tattoo removal pen of the present invention, generally described as 102, including a head piece 108 containing a needle cartridge with at least one needle bundle, a fillable and refillable glass syringe containing removal fluid 114, a fillable and refillable glass syringe containing numbing fluid 116, a dual syringe pump 118, a Y-shaped fluid connector 134, and a touch screen 128. Advantageously the Y-shaped fluid connector 134 is operable to simultaneously deliver the removal fluid and the numbing fluid through the head piece 108 containing a needle cartridge of the tattoo removal pen.

FIG. 2F illustrates a second interior front view of a tattoo removal pen of the present invention generally described as 102, including a head piece 108 containing a needle cartridge with at least one needle bundle, a fillable and refillable glass syringe containing removal fluid 114, a fillable and refillable glass syringe containing numbing fluid 116, a dual syringe pump 118, a Y-shaped fluid connector 134, and a touch screen 128.

FIG. 2G illustrates an additional interior front view of a tattoo removal pen of the present invention generally described as 102, including an on/off button 126, a charging port 130, a charging AC power cord 182, a battery housing 196 containing at least one rechargeable battery and including a bottom twist-on contact power point magnet plate 194, a software housing 192 including a top twist on contact power point magnet plate 228 and a bottom screwed in contact power point magnet plate 190, a touch screen 128, a multiple micro machine housing 224 including a screwed in top contact power point magnet plate 226, a motor housing 180 containing a rotary motor 184 at least two micro stepper motors 178A/178B, a rotary motor grommet 186, a metal needle plunger bar 188 within a metal needle plunger bar housing 220, at least two infusion screws 230A/230B within infusion screw housings 174/176, at least two infusion screw pusher bolts 232A/232B, at least two infusion screw plunger plates 234A/234B, and at least two hollow syringe plungers 236A/236B. The tattoo removal pen 102 further includes a syringe housing 228 including a fillable/refillable glass syringe for removal fluid 114 and a fillable/refillable glass syringe for numbing fluid 116, a removal fluid refill hole 132, a numbing fluid refill hole 136, an infusion screw housing 174/176, at least two male syringe ports 172A/172B, at least two female Y ports 170A/170B, a Y-shaped fluid connector 134, a sealed Y-shaped fluid connector female port 168, a mixing agitator 166, a metal needle plunger bar end stopper 164, a male hollow needle bundle plunger housing port 162, a head piece 108 comprising a top head piece 150 and a bottom head piece 152, a tamper proof chip 242, a needle bundle syringe topper 156, a needle plate 158, a needle bundle 154, and a grip 160.

In one embodiment, the battery housing 196 includes at least one rechargeable battery. In one embodiment, the software housing 192 includes at least one software chip, wherein the at least one software chip is housed in the back software housing 192 of the tattoo removal pen 102.

In a preferred embodiment, the multiple micro machine housing 224 includes a motor housing 180. In one embodiment, the motor housing 180 includes at least two motor housing wire ports 238A/238B. In one embodiment, the at least two motor housing wire ports 238A/238B are in the top of the motor housing 180. In one embodiment, the screwed in top contact power point magnet plate 226 of the multiple micro machine housing 224 includes at least two wire ports 240A/240B, wherein the at least two wire ports 240A/240B allow at least one wire to pass through to connect to a control plate.

In an alternate embodiment, the multiple micro machine housing 224 includes a rotary motor housing and at least two micro stepper motor housings. In one embodiment, the rotary motor 184 includes a rotary motor wire. In one embodiment the rotary motor housing 182 includes at least five wire holes, wherein two wire holes are on the top of the multiple micro machine housing 224 which connect to the screwed in top contact power point magnet plate 226, wherein two wire holes are on the bottom of the rotary motor housing 182 and two wire holes are on the top of the rotary motor housing 182. In one embodiment, each stepper motor housing 180A/180B includes at least one wire hole at the top of the stepper motor housing 180A/180B. In one embodiment, each stepper motor 178A/178B includes a stepper motor wire.

In one embodiment, the rotary motor housing 182 houses the rotary motor 184 and the rotary motor grommet 186. In one embodiment, the multiple micro machine housing 224 includes at least two wire port holes operable to route a rotary motor wire to the software housing 192 for connection to the battery within the battery housing 196. In one embodiment, the rotary motor 184 is operable to operate the needle plunger bar 188, which controls the speed, throw, and needle depth of the needle bundle 154. In one embodiment, the rotary motor grommet 186 is attached to the needle plunger bar 188 and operable to operate the needle bundle bar 188. In one embodiment, the needle plunger bar 188 is housed within a needle plunger bar housing 220. In one embodiment, the needle plunger bar 188 is operable to touch and push the plunger of the head piece 108.

In one embodiment, the needle plate 158 is operable to secure the needle bundle 154 in place and in a star-pattern.

In one embodiment, the at least two stepper motor housings 180A/180B house the at least two micro stepper motors 178A/178B. In one embodiment, the at least two micro stepper motors 178A/178B each include a micro stepper wire, which is routed through wire port holes of the multiple micro machine housing 224 to connect to the top contact power point magnet plate 226 of the multiple micro machine housing 224.

In one embodiment, the syringe housing 228 includes at least one glass window which exposes the syringe containing the tattoo removal fluid 114 and the syringe containing the numbing fluid 116. In one embodiment, the glass window further exposes the Y-shaped fluid connector 134. In one embodiment, the glass window includes at least two glass hinges and a glass door latch, advantageously providing for the ability to remove the syringe containing the tattoo removal fluid 114 and the syringe containing the numbing fluid 116 for cleaning and sanitizing. In one embodiment, the Y-shaped fluid connector 134 is made of glass, providing the ability to remove the Y-shaped fluid connector 134 for cleaning and sanitizing.

In one embodiment, the entire syringe housing 228 is made of glass. In one embodiment, the entire syringe housing 228 and the head piece 108 are made of glass. In an alternative embodiment, the head piece 108 includes at least one glass window, providing visibility to ensure proper connection of internal parts of the tattoo removal pen 102. In one embodiment, the head piece 108 includes a tamper proof chip 242 is operable to record and report inventory usage and prevents reuse of a head piece 108.

In one embodiment, the on/off button 126 is operable to turn the tattoo removal pen 102 on and/or off. In one embodiment, the charging port 130 is operable to connect to a wired electric power cord to charge the battery of the tattoo removal pen 102. In one embodiment, the tattoo removal pen 102 includes a battery housing 196, which includes a bottom twist on contact power point magnet plate 194 operable to connect the battery housing to the top contact power point magnet plate 228 of the software housing 192 of the tattoo removal pen 102. In one embodiment, the battery housing 196 is operable to twist on and connect to the software housing 192 of the tattoo removal pen 102. In one embodiment, the top contact power point magnet plate 228 of the software housing 192 is screwed on to the software housing 192, advantageously providing the ability to unscrew and take apart the tattoo removal pen 102 for any software related issues.

In one embodiment, the software housing 192 of the tattoo removal pen 102 includes at least one chip. In one embodiment, the software housing 192 includes a bottom contact power point magnet plate 190 to connect to the top contact power point magnet plate 226 of the multiple micro machine housing 224. In one embodiment, the bottom contact power point magnet plate 190 of the software housing 192 is screwed on to the software housing 192.

In one embodiment, the at least two female Y ports 170A/170B are operable to connect to the at least two male syringe ports 172A/172B, thereby connecting the fillable/refillable glass syringe containing removal fluid 114 and a fillable/refillable glass syringe containing numbing fluid 116 to the Y-shaped fluid connector 134. In one embodiment, the at least two male syringe ports 172A/172B are operable to control even flow of the fluids, including the removal fluid and the numbing fluid into the Y-shaped fluid connector 134. In one embodiment, the end of the metal needle bar housing 220 tightly seals into the middle of the inside of the Y-shaped fluid connector female port 168. In one embodiment, the Y-shaped fluid connector female port 168 is operable to allow the needle plunger bar 188 to pass through the Y-shaped fluid connector 134. Advantageously, the Y-shaped fluid connector female port 168 fully seals the Y-shaped fluid connector 134, as to not permit any fluid back up into the device.

In one embodiment, the mixing agitator 166 is located at the bottom of the Y-shaped fluid connector 134 allowing for two different, separate fluids to pass through and mix together before existing the tattoo removal pen 102. In one embodiment, the mixing agitator 166 is operable to mix the removal fluid and the numbing fluid. In one embodiment, gravity and the vibration of the tattoo removal pen 102 aid the mixing agitator 166 in mixing each fluid together. In one embodiment, the mixed fluids flow out of the tattoo removal pen 102 through the head piece 108 and over the needles of the needle bundle 154, advantageously allowing the fluids to drip slowly onto a treatment area of skin.

In one embodiment, the metal needle plunger bar end stopper 164, is operable to stop the needle plunger bar 188 and prevent the needle plunger bar 188 from being pushed too low to the point that it damages the head piece 108.

In one embodiment, the male hollow needle bundle plunger housing port 162 is operable to permit the mixed fluids through and maintain the fluids in the needle plunger bar housing 220, flowing from the Y-shaped fluid connector 134 to the head piece 108.

In one embodiment, the grip 160 is a thin rubber grip, operable to provide greater grip ability for a technician to hold the tattoo removal pen 102. In one embodiment, the grip 160 is a thin silicone grip, operable to provide greater grip ability for a technician to hold the tattoo removal pen 102. In one embodiment, the grip 160 is operable to encourage proper hand placement and/or hand comfort for a technician. In one embodiment, the grip 160 is attached to the head piece 108.

In one embodiment, the head piece 108 includes a hollow syringe topper 156, operable to permit at least one fluid to drop over top of the needles in the needle bundle. In one embodiment, the head piece 108 includes a hollow syringe topper 156, operable to allow the removal fluid and/or the numbing fluid to drop over top of the needles in the needle bundle 154.

In one embodiment, the head piece 108 of the tattoo removal pen 102 includes a cartridge housing, a needle plunger, and a needle bundle. In one embodiment, the cartridge housing is operable to be solid in color. In one embodiment, the cartridge housing is operable to be clear in color. In one embodiment, the head piece 108 includes a needle membrane operable to prevent the tattoo removal fluid and/or the numbing fluid from flowing back into the tattoo removal pen. In one embodiment, the needle membrane is made of silicone.

In one embodiment the tattoo removal pen 102 is made of plastic. In one embodiment the tattoo removal pen 102 is made of silicone. In one embodiment the tattoo removal pen 102 is made of metal. In one embodiment the tattoo removal pen 102 is made of stainless steel. In one embodiment the tattoo removal pen 102 is made of aluminum. In one embodiment the tattoo removal pen 102 is made of a medical grade material.

FIG. 2H illustrates an interior front view of the multiple micro machine housing 224 and the syringe housing 228 of the tattoo removal pen, according to one embodiment of the present invention, including motor housing 180, at least two motor housing wire ports 238A/238B, a rotary motor 184, a rotary motor grommet 186, a needle plunger bar 188, a needle plunger bar housing 220,, at least two micro stepper motors 178A/178B, at least two infusion screws 230A/230B within infusion screw housings 174/176, at least two infusion screw pusher bolts 232A/232B, at least two infusion screw plunger plates 234A/234B, at least two hollow syringe plungers 236A/236B, a syringe housing 228 including a fillable/refillable glass syringe containing removal fluid 114 and a fillable/refillable glass syringe containing numbing fluid 116, a removal fluid refill hole 132, a numbing fluid refill hole 136, at least two male syringe ports 172A/172B, at least two female Y ports 170A/170B, a Y-shaped fluid connector 134, a sealed Y-shaped fluid connector port 168.

In one embodiment, each micro stepper motor 172A/178B of the at least two micro stepper motors 178A/178B are operable to operate an infusion screw 230A/230B. In one embodiment, an infusion screw 230A/230B is contained within an infusion screw housing 174/176. In one embodiment, the infusion screw housing 174/176 goes all the way down to the bottom of the glass syringe. In one embodiment, the infusion screws 230A/230B are operable to spin counter-clockwise within the infusion screw housing 174/176, which pushes an infusion screw pusher bolt 232A/232B against the infusion screw plunger plate 234A/234B. In one embodiment, the infusion screws 230A/230B are operable to spin clockwise within the infusion screw housing 174/176, which pushes an infusion screw pusher bolt 232A/232B against the infusion screw plunger plate 234A/234B. In one embodiment, each infusion screw 230A/230B includes at least one infusion screw pusher bolt 232A/232B. In one embodiment, the infusion screw plunger plate 234A/234B includes at least one hole in the middle of the plate, allowing the plate to slide and seal around the housing for the infusion screws 230A/230B. In one embodiment, the infusion screw plunger plate 234A/234B is connected to a hollow syringe plunger 236A/236B, wherein the hollow syringe plunger 236A/236B is operable to surround the housing for the infusion screws.

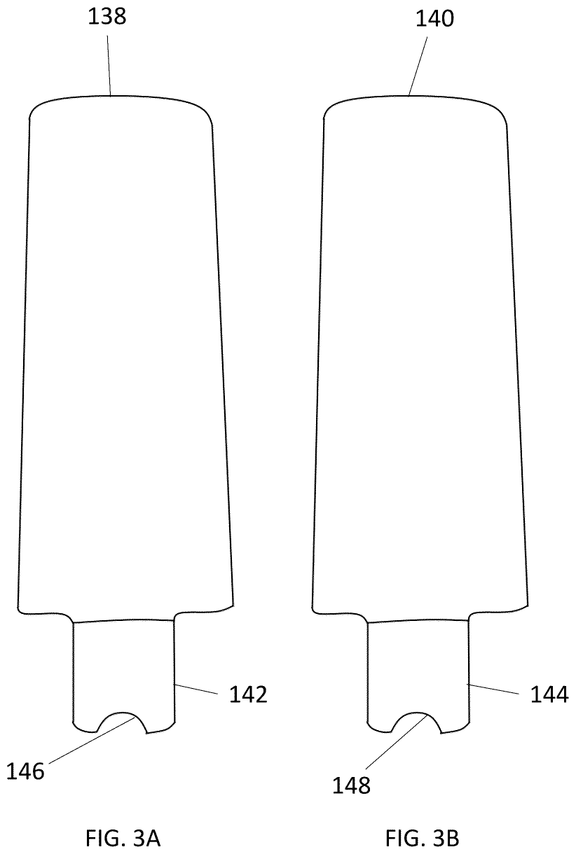

FIG. 3A illustrates a tattoo removal fluid canister 138, including a refill valve 142 and a female connector 146. In one embodiment, female connector 146 is operable to connect and lock into the tattoo removal pen 102. In one embodiment, the tattoo removal fluid canister 138 includes a measuring window beaker. FIG. 3B illustrates a numbing fluid canister 140, including a refill valve 144 and a female connector 148. In one embodiment, female connector 148 is operable to connect and lock into the tattoo removal pen 102. In one embodiment, the numbing fluid canister 140 includes a measuring window beaker. In one embodiment, a tattoo removal fluid canister 138 and/or a numbing fluid canister 140 are operable to attach to a wall mount to be hung on a wall, thereby providing easy access for refilling removal fluid and/or numbing fluid.

In one embodiment, the touch screen of the tattoo removal machine and the screen of the tattoo removal pen are operable to allow for a user to touch the screen to conduct a plurality of different functions of the tattoo removal machine software. In one embodiment, the touch screen of the tattoo removal machine and the screen of the tattoo removal pen are operable to show a client chart and a client's vitals. In one embodiment, client vital data is obtained from a plurality of sensors connected to the client. In one embodiment, the plurality of sensors includes a heart rate sensor, blood pressure sensor, electrocardiogramsor, pressure sensor, accelerometer sensor, fluid level sensor, body temperature sensor, galvanic skin response (GSR) sensor, respiratory rate sensor, and/or pulse oximeter sensor. In one embodiment, the client vital data is taken before a procedure and manually input into the tattoo removal machine by a technician. In one embodiment, the touch screen of the tattoo removal machine and the screen of the tattoo removal pen are operable to show client health categories, including activity, body measurements, heart, medications, mobility, mental wellbeing, and/or nutrition. In one embodiment, client data relating to the client health categories is manually input into the tattoo removal machine by the technician. In one embodiment, the client data relating to the client health categories is uploaded electronically to a user profile by the client, wherein the tattoo removal machine software is operable to connect to the user profile.

In one embodiment, the touch screen of the tattoo removal machine and the screen of the tattoo removal pen are operable to show the needle depth and speed of the tattoo removal pen during a procedure. In one embodiment, the tattoo removal pen is operable to visually show the needle depth on the tattoo removal pen. In one embodiment, the tattoo removal pen is operable to include heat sensors to detect and measure the needle depth. In one embodiment, the touch screen of the tattoo removal machine and the screen of the tattoo removal pen are operable to show the tattooed areas of the client's body that are undergoing a procedure live. In one embodiment, the touch screen of the tattoo removal machine and the screen of the tattoo removal pen are operable to show the level of removal fluid and numbing fluid remaining in the syringes in the tattoo removal machine, advantageously allowing for inventory tracking and determining how much removal fluid and numbing fluid is distributed to each client per procedure. In one embodiment, the touch screen of the tattoo removal machine and the screen of the tattoo removal pen are operable to show the removal procedures and protocols.

In one embodiment, the software of the tattoo removal machine and the tattoo removal pen is operable to control how the dual syringe pump, the tattoo removal pen, and the tattoo removal machine function together. In one embodiment, the software of the tattoo removal machine and the tattoo removal pen includes pre-programmed protocols for the tattoo removal pen and removal settings and protocols.

In one embodiment, the software of the tattoo removal machine and the tattoo removal pen updates automatically via Over the Air (OTA) updates or other wireless updates. In one embodiment, the software of the tattoo removal machine and the tattoo removal pen updates after a manual update implemented by at least one assigned user per center.

In one embodiment, the software of the tattoo removal machine and the tattoo removal pen measures the removal fluid and the numbing fluid based on pre-programmed standard protocols. In one embodiment, the removal protocols include the standard method steps and procedures using the system of the present invention. In one embodiment, the pre-programmed protocols include protocols for the entire system of the present invention, including the tattoo removal pen and the dual syringe pump. In one embodiment, the pre-programmed protocols include protocols for the depth a needle can puncture the skin of a client. In one embodiment, the software of the tattoo removal machine is operable to generate an alert if the needle depth is programmed too deep or on too fast of a speed, advantageously preventing damage to a client's skin. In one embodiment, the pre-programmed protocols include protocols for the speed of the tattoo removal pen. In one embodiment, the pre-programmed protocols are operable to be overridden by a technician. In one embodiment, the software of the tattoo removal machine is operable to translate instructions into a plurality of different languages.

In one embodiment, the software of the tattoo removal machine and the tattoo removal pen is operable to automatically produce an alert when either the numbing fluid or removal fluid reach a pre-set low level. In one embodiment, the software of the tattoo removal machine and the tattoo removal pen is operable to automatically produce an alert based on needle depth and/or speed of the tattoo removal pen. In one embodiment, the software of the tattoo removal machine and the tattoo removal pen is operable to automatically produce an alert based on a needle depth of the tattoo removal pen being above a maximum determined threshold. In one embodiment, the software of the tattoo removal machine and the tattoo removal pen is operable to automatically produce an alert based on a needle depth of the tattoo removal pen being below a minimum determined threshold. In one embodiment, the software of the tattoo removal machine and the tattoo removal pen is operable to automatically produce an alert based on a speed of the tattoo removal pen exceeding a determined maximum speed threshold. In one embodiment, the software of the tattoo removal machine and the tattoo removal pen is operable to automatically produce an alert based on a speed of the tattoo removal pen being below a determined minimum speed threshold. In one embodiment, the software of the tattoo removal machine and the tattoo removal pen is operable to produce an alert based on client vitals reaching or exceeding pre-determined values. In one embodiment, the software of the tattoo removal machine and the tattoo removal pen is operable to produce an alert based on improper technique, such as the positioning, angle, and/or pressure of the tattoo removal pen. In one embodiment, the alert is an audio output sound. In one embodiment, the alert is a visual alert on the touch screen of the tattoo removal machine or the screen of the tattoo removal pen. In one embodiment, the alert is an automatic shutoff of the tattoo removal machine. In one embodiment, the alert is an automatic shutoff of the tattoo removal pen.

In one embodiment, the software of the tattoo removal machine and the tattoo removal pen is operable to include a silent alarm for potential human trafficking victims to signal if a client is afraid to leave, has not escaped their abuse, the abuser is with the client at the appointment, and/or needs help. In one embodiment, the silent alarm contacts the authorities, including the police, paramedic, and/or firefighters.

In one embodiment, the software of the tattoo removal machine and the tattoo removal pen records every procedure, including procedure timing, amount of removal fluid used, amount of numbing fluid used, protocols followed, divergence from set protocols, client information, client demographics, client vitals, client preferences, settings used during the procedure, technician identity, technician-specific settings, and/or other supplies used in the procedure. In one embodiment, specific notes about any previous procedures the client has previously undergone will be documented by a technician and input into the software of the tattoo removal machine, wherein the notes on previous procedures include multiple-choice question answers and/or personal notes. In one embodiment, the multiple-choice question answers can include healing results, deviations from aftercare, complications and/or successful positive results. In one embodiment, if poor results or allergic reactions are reported, the tattoo removal machine or the tattoo removal pen is operable to prohibit the procedure from proceeding. In one embodiment, the tattoo removal machine or the tattoo removal pen is operable to prohibit a new procedure from starting if the previous procedure notes and previous client charts are not complete.

In one embodiment, the software of the tattoo removal machine and the tattoo removal pen is operable to track statistics of procedures, including supplies used in a procedure, to assist with inventory. In one embodiment, the software of the tattoo removal machine and the tattoo removal pen is operable to allow technicians to input supply orders via the touch screen of the tattoo removal machine or via the screen of the tattoo removal pen. In one embodiment, the software of the tattoo removal machine and the tattoo removal pen is operable to allow technicians or at least one user to view the existing inventory of supplies, removal fluid, numbing fluid, and/or technician equipment.

In one embodiment, the software of the tattoo removal machine and the tattoo removal pen is operable to monitor and track statistics and specific details of tattoo removal procedures. In one embodiment, the software of the tattoo removal machine and the tattoo removal pen is operable to track revenue of tattoo removal procedures.

In one embodiment, the software of the tattoo removal machine and the tattoo removal pen is operable to include universal training protocols, supplies inventory, online orders, client records, client procedure charts, client payment information, machine statistics, and/or the programming tool to update and troubleshoot all tattoo removal machines.

In one embodiment, the system of the present invention includes medical grade aftercare products for a client to use on the tattoo removal area post-procedure. In one embodiment, the aftercare products include a scar treatment silicone gel, a sheer mineral repair SPF 45 cream, a hydrocortisone and aloe vera anti-itch cream, and/or a soothing recovery ointment.

In an alternative embodiment, the tattoo removal pen is operable to include a multi-function light module, wherein the multi-function light module is operable to provide illumination/light and provide visual feedback through a blinking light and/or light color changes. In one embodiment, the multi-function light module is operable to provide visual alerts for one or more conditions, including, but not limited to excessive needle depth, excessive pressure, incorrect angle orientation, fluid depletion and/or low fluid levels, and/or flow status for the numbing fluid, removal fluid, or both fluids. In one embodiment, the visual alert is a flashing light, such as a flashing red light. In one embodiment, the visual alert is a light color change, such as a change from a green light to a red light. In one embodiment, the visual alert is synchronized with audible alerts.

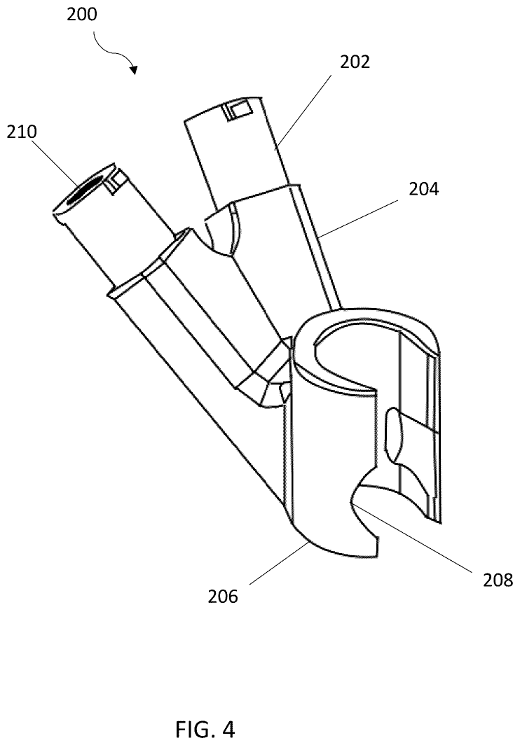

FIG. 4 illustrates a first front perspective view of a dual Y clip of the present invention, generally described as 200, having an upper Y stem 202, a lower Y stem 204, a clip 206, a curvature on the clip 208, and a channel 210.

In one embodiment, the dual Y clip 200 is y-shaped with two stems, including an upper Y stem 202 and a lower Y stem 204 on both stems of the Y clip. In one embodiment, the upper Y stem 202 is operable to retract into the lower Y stem 204. In one embodiment, the stems of the dual Y clip 200 are oriented at an approximate 36-degree angle from each other. In one embodiment, the stems of the dual Y clip 200 are at least 36 degrees from each other. In one embodiment, the stems of the dual Y clip 200 are less than 36 degrees from each other. In one embodiment, the stems of the dual Y clip 200 are more than 36 degrees from each other. In one embodiment, the dual Y clip 200 has a smooth exterior texture. In one embodiment, the dual Y clip 200 has a rough exterior texture.

In one embodiment, the curvature on the clip 208 is operable to matingly pair and connect the Y clip to the tattoo removal pen of the present invention. In one embodiment, the curvature on the clip 208 is operable to allow the Y clip to attach to a plurality of different shaped tattoo removal pens.

In one embodiment, the channel 210 provides the opening for the IV lines to insert into the Y clip 200. In one embodiment, the IV lines are inserted into the channel 210 of the Y clip 200.

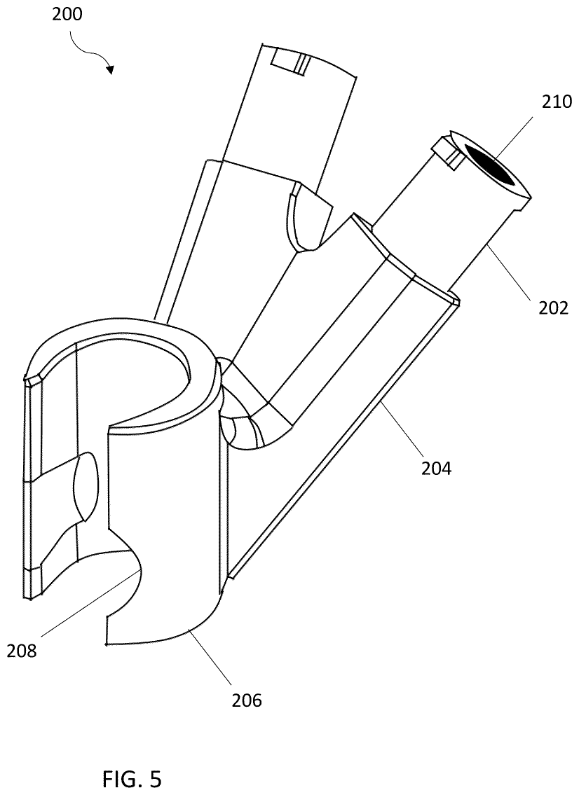

FIG. 5 illustrates a second front perspective view of a dual Y clip of the present invention, generally described as 200, having an upper Y stem 202, a lower Y stem 204, a clip 206, a curvature on the clip 208, and a channel 210.

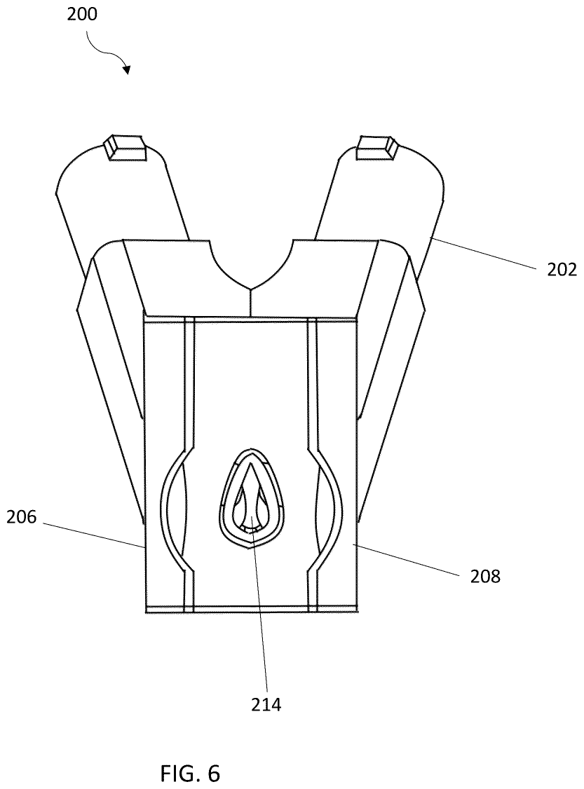

FIG. 6 illustrates a front orthogonal view of a dual Y clip of the present invention, generally described as 200, having an upper Y stem 202, a clip 206, a curvature on the clip 208, and a port 214. In one embodiment, the removal fluid and the numbing fluid simultaneously flow through the port 214 and onto a client's skin for the removal of a tattoo and client comfort during the tattoo removal.

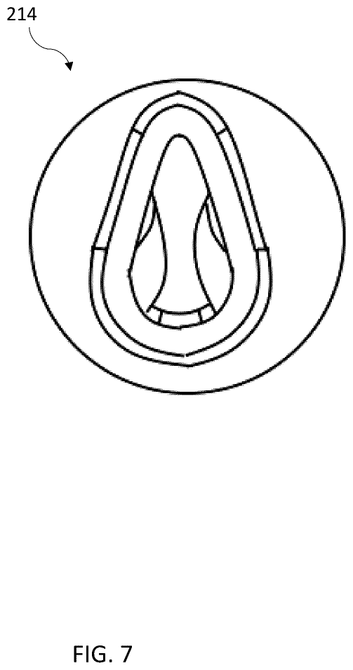

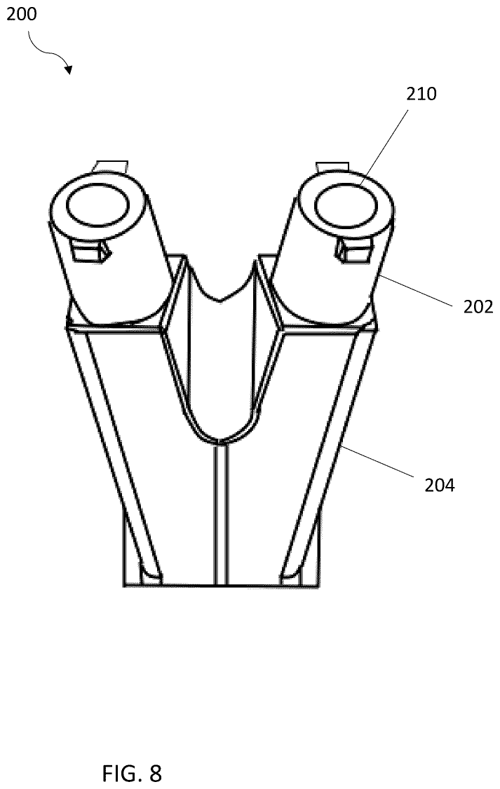

FIG. 7 illustrates an enlarged view of a port of the dual Y clip of the present invention, generally described as 214. In one embodiment, the removal fluid and the numbing fluid flow through the port 214 and into the head piece containing a needle cartridge of the tattoo removal pen through an opening in the side wall of the head piece.

FIG. 8 illustrates a back orthogonal view of a dual Y clip of the present invention, generally described as 200, having an upper Y stem 202, a lower Y stem 204, and a channel 210.

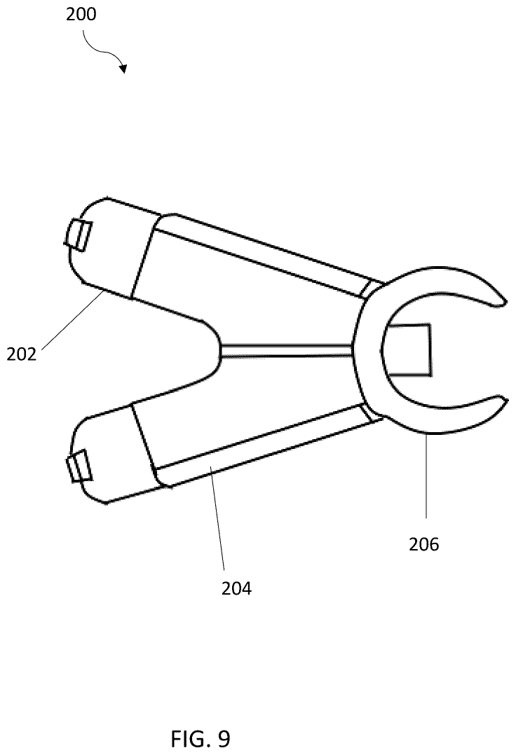

FIG. 9 illustrates a top orthogonal view of a dual Y clip of the present invention, generally described as 200, having an upper Y stem 202, a lower Y stem 204, and a clip 206.

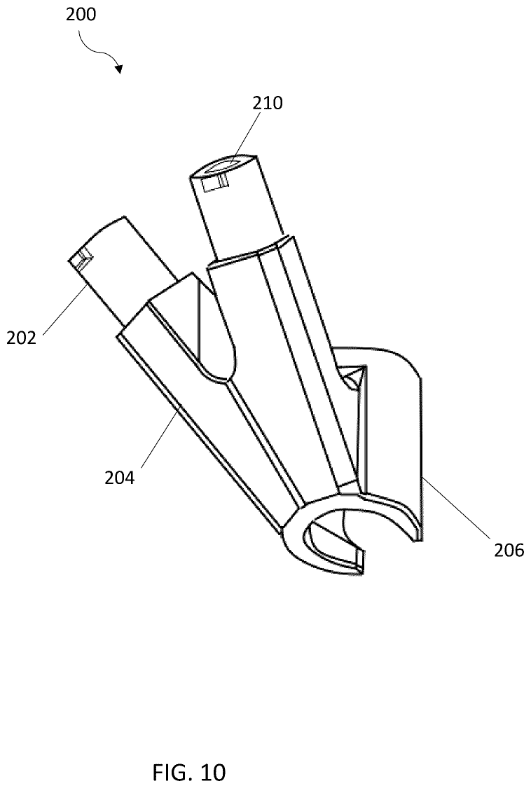

FIG. 10 illustrates a back perspective view of a dual Y clip of the present invention, generally described as 200, having an upper Y stem 202, a lower Y stem 204, a clip 206, and a channel 210.

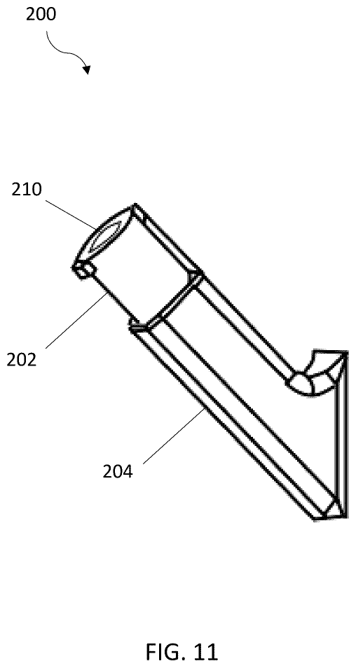

FIG. 11 illustrates a side orthogonal view of a dual Y clip stem of the present invention, generally described as 200, having an upper Y stem 202, a lower Y stem 204, and a channel 210.

In one embodiment, the Y clip is operable to change color after being used, advantageously preventing re-use of the same Y clip for another procedure and/or another client.

In one embodiment, the Y clip is operable to include a cap on each stem of the Y clip, wherein the cap is operable to close one stem of the Y clip if only one fluid is being dispensed through the Y clip, advantageously preventing the passage of blood-borne pathogens.

In one embodiment, the system of the present invention is operable to remove a tattoo or permanent makeup in as little as one session. In one embodiment, the system of the present invention is operable to remove a tattoo or permanent makeup in at least one session. In one embodiment, the system of the present invention is operable to remove a tattoo or permanent makeup after multiple sessions.

In an alternative embodiment, the tattoo removal pen is operable to be modified for tattoo application by equipping the dual syringe compartments to include at least two separate tattoo ink colors, wherein the tattoo removal pen is operable to simultaneously administer two separate tattoo ink colors. Additionally, the dual syringe compartments provide for easy switching between colors mid-procedure without the need to dip or reload the needle.

In an alternative embodiment, the tattoo removal pen is operable to be used for administering therapeutic agents, including but not limited to, scar revision compounds, such as collagen, topical anesthetics, such as lidocaine, and/or regenerative treatments, such as peptides or platelet-rich plasms (PRP). In an alternative embodiment, the tattoo removal pen is operable to be used for administering therapeutic agents for scar recovery post-surgery or trauma. In an alternative embodiment, the tattoo removal pen is operable to be used for administering topical anesthetics for dermatological procedures. In an alternative embodiment, the tattoo removal pen is operable to be used for administering at least one compound during a cosmetic procedure, including the micro-infusion of dual-action compounds.

In an alternative embodiment, the tattoo removal machine is operable to treat an entire tattooed area of skin at the same time. In an alternative embodiment, the present invention provides a tattoo removal machine operable to scan a tattooed area of skin, wherein based on the scan, the tattoo removal machine is operable to automatically determine the shape, area, and/or perimeter of the tattooed area of skin. In one embodiment, the tattoo removal machine is operable to automatically determine the depth of the area of tattooed skin. In one embodiment, based on the determined shape, area, perimeter, and/or depth of the tattooed area, the tattoo removal machine is further operable to determine the exact number and length of needle bundles required to treat the entire tattooed area at the same time. In one embodiment, the tattoo removal machine includes a needle plate containing a plurality of needle bundles. In one embodiment, the needle plate is disposable. In one embodiment, the needle plate is a one-time use needle plate and operable to be switched out with a new needle plate after a procedure. In one embodiment, the tattoo removal fluid and the numbing fluid are distributed behind the needle plate, such that the tattoo removal fluid and the numbing fluid are administered through the needles of the needle bundles. In one embodiment, the needle bundles are operable to abrade the skin by oscillating along determined treatment area. In one embodiment, a tattoo removal fluid in is operable to soften the epidermis layer of skin, allowing the oscillating needle bundle to more efficiently create a small surface wound, which allows the ink of a tattoo to draw to the surface of the skin. The small surface wound created by the needle bundle creates an opening for the tattoo ink pigment to rise to the surface of the skin when exposed to oxygen, collecting on the underside of a scab which forms over the small surface wound. In an alternative embodiment, the needles of the needle bundle are hollow, such that the needles are operable to administer a tattoo removal fluid and/or a numbing fluid. In one embodiment, the needle bundles are operable to administer a tattoo removal fluid and/or a numbing fluid simultaneously.

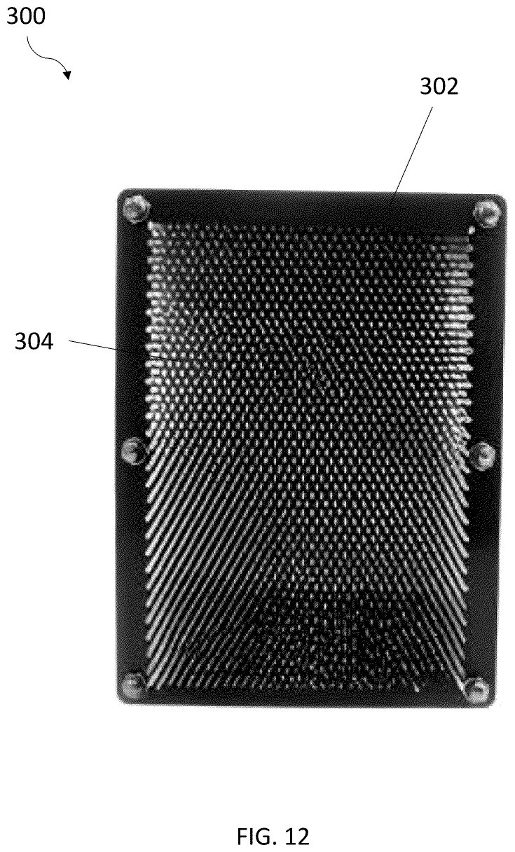

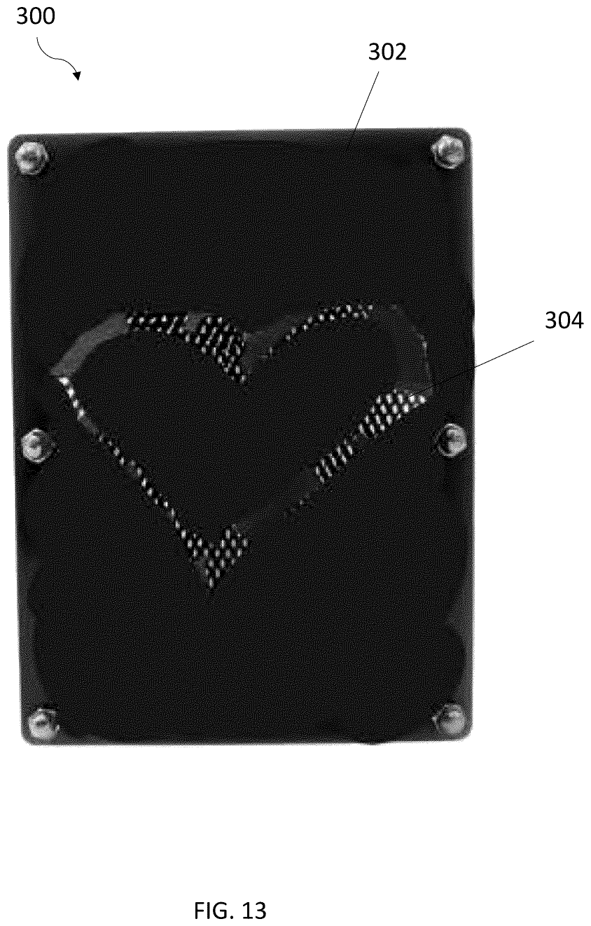

FIG. 12 illustrates a tattoo removal machine 300 including a needle plate 302 with a plurality of needles 304. FIG. 13 illustrates a tattoo removal machine 300 including a needle plate 302 with a plurality of needles 304 configured in the shape of a tattoo to be removed.

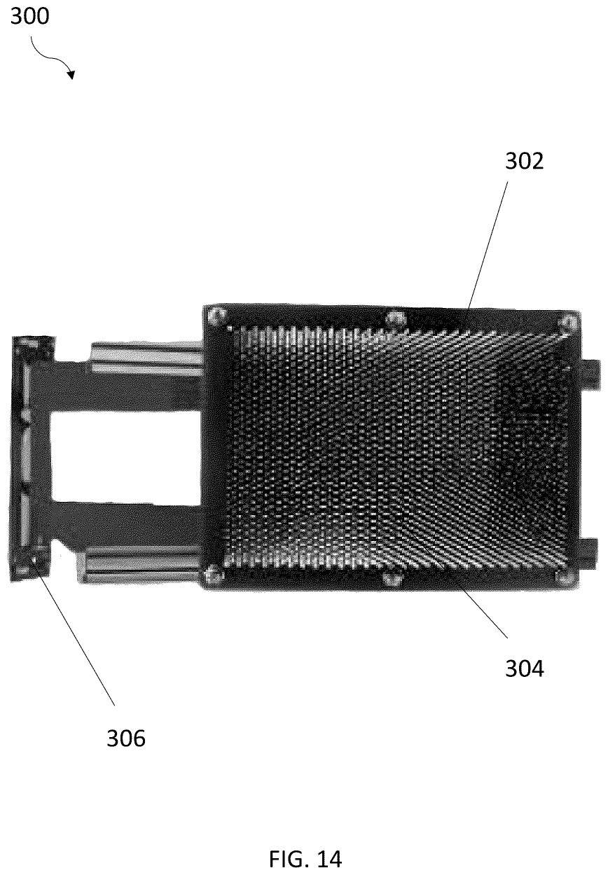

FIG. 14 illustrates a tattoo removal machine 300 including a needle plate 302 with a plurality of needles 304 and a wall mount 306. In one embodiment, the tattoo removal machine 300 is operable to attach to a wall mount 306 for hanging on a wall.

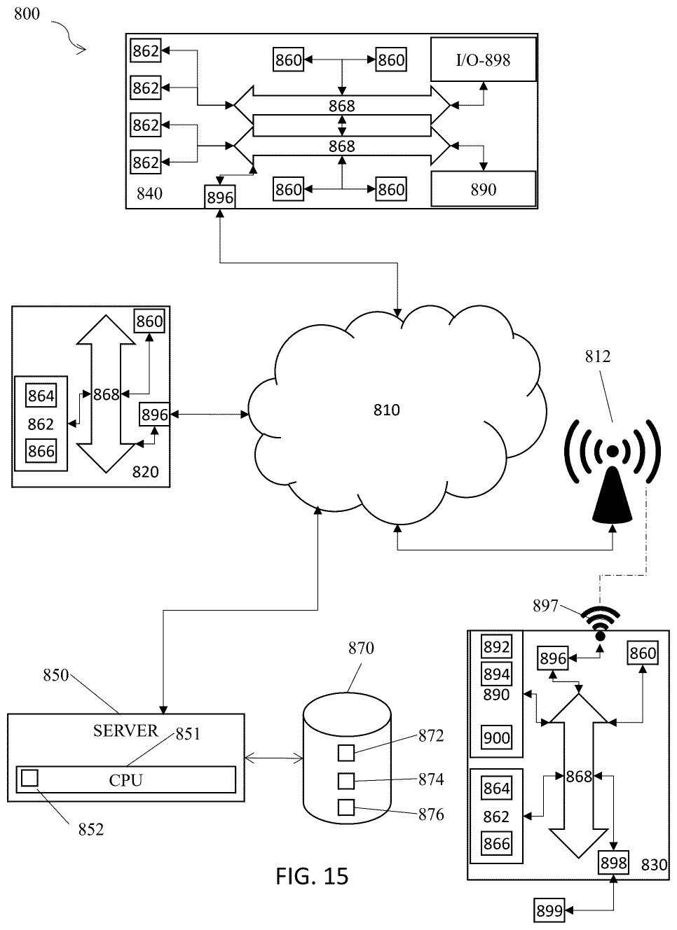

FIG. 15 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, gaming controllers, joy sticks, touch pads, signal generation devices (e.g., speakers), augmented reality/virtual reality (AR/VR) devices (e.g., AR/VR headsets), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 15, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 15, is operable to include other components that are not explicitly shown in FIG. 15 or is operable to utilize an architecture completely different than that shown in FIG. 15. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A device for laser-free tattoo removal, comprising:
a head piece comprising a needle cartridge including at least one needle bundle;
at least one fillable glass syringe containing tattoo removal fluid;
at least one second fillable glass syringe containing numbing fluid;
an internal dual syringe pump; and
a Y-shaped fluid connector;
wherein the internal dual syringe pump is operable to administer the tattoo removal fluid and the numbing fluid by pushing a plunger of the at least one fillable glass syringe containing tattoo removal fluid and by pushing a plunger of the at least one second fillable glass syringe containing numbing fluid;
wherein the internal dual syringe pump is operable to control the flow rates of the tattoo removal fluid and the numbing fluid, such that the tattoo removal fluid and the numbing fluid are operable to be administered at the same or different flow rates;
wherein the Y-shaped fluid connector is operable to connect the tattoo removal fluid and the numbing fluid;
wherein the tattoo removal fluid and the numbing fluid are delivered through the head piece; and
wherein the device is operable to administer the tattoo removal fluid and the numbing fluid through the head piece simultaneously.

2. The device of claim 1, wherein the device is operable to abrade a first treatment area of skin by creating by plurality of small shapes with the at least one needle bundle on the first treatment area of skin.

3. The device of claim 1, wherein the device further includes at least one screen, wherein the at least one screen is a touch screen, wherein the screen is operable to display the depth of the at least one needle bundle when the device is in use, wherein the screen is further operable to display the device speed when the device is in use.

4. The device of claim 1, wherein the at least one fillable glass syringe containing the tattoo removal fluid is refillable, wherein the at least one fillable glass syringe containing the tattoo removal fluid includes at least one tattoo removal fluid refill hole, wherein the at least one second fillable glass syringe containing the numbing fluid is refillable, wherein the at least one second fillable glass syringe containing the numbing fluid includes at least one numbing fluid refill hole.

5. The device of claim 1, wherein the device is operable to wirelessly connect to an external device.

6. The device of claim 1, wherein the device includes software, wherein the software is operable to be updated through a wireless update.

7. The device of claim 1, wherein the device is operable to measure the amount of tattoo removal fluid and/or numbing fluid, wherein the device is operable to produce an automatic alert if the amount of tattoo removal fluid and/or the amount of the numbing fluid fall below a set threshold level.

8. The device of claim 6, wherein the software is operable to automatically shutoff the device.

9. A method of removing a tattoo from a human patient using a laser-free device, comprising:
determining a first treatment area of skin of the human patient;
marking a perimeter of the first treatment area of skin of the human patient with a surgical marker;
abrading the first treatment area of skin with at least one needle bundle of the laser-free device by creating a plurality of small shapes with the at least one needle bundle on the first treatment area of skin; and
delivering, with a head piece of the laser-free device, a tattoo removal fluid to the first treatment area of skin of the human patient while simultaneously delivering, with the head piece of the laser-free device, a numbing fluid to the first treatment area of skin of the human patient.

10. The method of claim 9, wherein the method does not include templating the first treatment area.

11. The method of claim 9, further comprising spacing at least one small shape of the plurality of small shapes at least 4mm apart from a second at least one small shape of the plurality of small shapes.

12. The method of claim 9, further comprising administering the tattoo removal fluid and the numbing fluid by an internal dual syringe pump of the laser-free device.

13. The method of claim 12, further comprising pushing, by the internal dual syringe pump, a plunger of a fillable glass syringe containing tattoo removal fluid, and pushing, by the internal dual syringe pump, a plunger of a fillable glass syringe containing numbing fluid.

14. The method of claim 12, further comprising controlling, by the internal dual syringe pump, the flow rates of the tattoo removal fluid and the numbing fluid, such that the tattoo removal fluid and the numbing fluid are operable to be administered at the same or different flow rates.

15. The method of claim 9, further comprising determining a depth of the at least one needle bundle required for removing the tattoo.

16. A device for laser-free tattoo removal, comprising:
a head piece comprising a needle cartridge including at least one needle bundle;
at least one fillable glass syringe containing tattoo removal fluid;
at least one second fillable glass syringe containing numbing fluid;
at least two stepper motors; and
a touch screen;
wherein the at least one fillable glass syringe containing the tattoo removal fluid is refillable, wherein the at least one fillable glass syringe containing the tattoo removal fluid includes at least one tattoo removal fluid refill hole;
wherein the at least one second fillable glass syringe containing the numbing fluid is refillable, wherein the at least one second fillable glass syringe containing the numbing fluid includes at least one numbing fluid refill hole;
wherein a stepper motor of the at least two stepper motors is operable to administer the tattoo removal fluid;
wherein a second stepper motor of the at least two stepper motors is operable to administer the numbing fluid;
wherein at least two stepper motors are operable to control the flow rates of the tattoo removal fluid and the numbing fluid, such that the tattoo removal fluid and the numbing fluid are operable to be administered at the same or different flow rates;
wherein the touch screen is operable to display a depth of the at least one needle bundle, wherein the touch screen is further operable to display a speed of the device;
wherein the tattoo removal fluid and the numbing fluid are delivered through the head piece; and
wherein the device is operable to administer the tattoo removal fluid and the numbing fluid through the head piece simultaneously.

17. The device of claim 16, wherein the device is operable to abrade a first treatment area of skin by creating by plurality of small shapes with the at least one needle bundle on the first treatment area of skin.

18. The device of claim 16, wherein the device includes a Y-shaped fluid connector and a mixing agitator, wherein the Y-shaped fluid connector is operable to connect the tattoo removal fluid and the numbing fluid, wherein the mixing agitator is operable to mix the tattoo removal fluid and the numbing fluid together.

19. The device of claim 16, wherein the device is operable to measure the amount of tattoo removal fluid and/or numbing fluid, wherein the device is operable to produce an automatic alert if the amount of tattoo removal fluid and/or the amount of the numbing fluid fall below a set threshold level.

20. The device of claim 16, wherein the at least one needle bundle includes at least five needles arranged in a star-shaped pattern.
